(19) United States
(12) Patent Application Publication
(10) Pub. No.: US 2011/0218143 A1
(43) Pub. Date: Sep. 8, 2011

(54) COMPOSITIONS AND METHODS FOR TISSUE REPAIR

(75) Inventors: Shalesh Kaushal, Gainesville, FL (US); Mani Annamalai, Gainesville, FL (US)

(73) Assignee: UNIVERSITY OF FLORIDA RESEARCH FOUNDATION, Gainesville, FL (US)

(21) Appl. No.: 12/743,907

(22) PCT Filed: Nov. 20, 2008
(86) PCT No.: PCT/US08/13000

§ 371 Date: May 23, 2011

Related U.S. Application Data
(60) Provisional application No. 61/003,785, filed on Nov. 20, 2007.
(60) Provisional application No. 61/003,787, filed on Nov. 20, 2007.
(60) Provisional application No. 61/003,786, filed on Nov. 20, 2007.
(60) Provisional application No. 61/003,824, filed on Nov. 20, 2007.
(60) Provisional application No. 61/083,070, filed on Jul. 23, 2008.

(51) Int. Cl. A61K 38/10; C12N 5/02; A61P 3/10; A61P 9/00; A61P 43/00; A61P 17/02; A61K 31/439; A61K 31/366; A61K 31/352; A61K 31/34; A61K 31/215; A61P 1/16; A61P 11/00; A61K 31/365

(52) U.S. Cl. 514/6.9; 435/375; 514/16.4; 514/16.5; 514/18.6; 514/21.5; 514/286; 514/453; 514/454; 514/468; 514/529

(57) ABSTRACT

The invention generally provides compositions and methods for the repair or regeneration of a tissue or organ in need thereof.

### CROSS-REFERENCE TO RELATED APPLICATION

This application claims the benefit of the following U.S. Provisional Application Nos. Ser. Nos. 61/003,786, filed Nov. 20, 2007; 61/003,787, filed Nov. 20, 2007; 61/003,785, filed Nov. 20, 2007; 61/003,824, filed Nov. 20, 2007; and 61/083,070, filed Jul. 23, 2008; the entire contents of each of which are incorporated herein by this reference.

### BACKGROUND OF THE INVENTION

Heart disease, diabetes, liver failure, and diseases or disorders characterized by tissue damage, increased cell death or a deficiency in cell number are not amenable to treatment with conventional therapies. The hematopoietic system generates the many different cell types that make up blood, but the commitment of HSCs to forming blood is not irreversible. A number of studies have shown that hematopoietic stem cells have the capacity to differentiate into alternate cell types, such as muscle cells (e.g., skeletal myocytes and cardiomyocytes), brain cells, liver cells, skin cells, lung cells, kidney cells, intestinal cells, and pancreatic cells. The number of HSCs having the potential to differentiate into alternate cell types represents a very small percentage of the total number of cells present in bone marrow. If the number of such cells could be increased, they might participate in the repair or regeneration of damaged or diseased tissues or organs. Methods of repairing damaged heart, pancreas, liver or other tissues are urgently required.

### SUMMARY OF THE INVENTION

As described below, the present invention provides compositions for tissue repair and bone marrow derived stem cell activation.

In one aspect, the invention provides a method for tissue repair or regeneration, the method involving contacting a cell with an effective amount of an agent having at least two, three, or four activities that is any one or more of i) inhibition of hsp-90 biological activity; ii) mobilization of a bone marrow derived stem cell; iii) inhibition of apoptosis; and iv) modulation of an immune response.

In another aspect, the invention provides a method for tissue repair or regeneration, the method involving contacting a cell with an effective amount of an agent that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or structural or functional analogs or derivatives of the agents.

In yet another aspect, the invention provides a method for tissue repair or regeneration in a subject, the method involving contacting a cell with an effective amount of an agent having at least two activities that is any one or more of i) inhibition of hsp-90 biological activity; ii) mobilization of a bone marrow derived stem cell; iii) inhibition of apoptosis; and iv) modulation of an immune response; activating a bone marrow derived stem cell of the subject; and recruiting the bone marrow derived stem cell to a tissue or organ in need of repair.

In still another aspect, the invention provides a method of treating a subject having a disease or disorder characterized by an undesirable increase in cell death or a deficiency in cell number, the method involving administering to the subject an effective amount of an agent having at least two activities that is any one or more of i) inhibition of hsp-90 biological activity; ii) mobilization of a bone marrow derived stem cell; iii) inhibition of apoptosis; and iv) modulation of an immune response, thereby treating the disease or disorder.

In another aspect, the invention provides a pharmaceutical composition for tissue repair or regeneration involving an effective amount of an agent having an activity that is any one or more of i) inhibition of hsp-90 biological activity; ii) mobilization of a bone marrow derived stem cell; iii) inhibition of apoptosis; and iv) modulation of an immune response in a pharmaceutically acceptable excipient.

In another aspect, the invention provides a pharmaceutical composition for tissue repair or regeneration involving an effective amount of an agent that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or functional or structural analogs thereof, in a pharmaceutically acceptable excipient. In one embodiment, the composition is a packaged pharmaceutical labeled for use in tissue repair or regeneration.

In another aspect, the invention provides a kit for tissue repair or regeneration, the kit containing an effective amount of an agent having an activity that is any one or more of i) inhibition of hsp-90 biological activity; ii) mobilization of a bone marrow derived stem cell; iii) inhibition of apoptosis; and iv) modulation of an immune response, and written instructions for using the kit.

In another aspect, the invention provides a kit for tissue repair or regeneration, the kit containing an effective amount of an agent that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and written instructions for using the kit.

In another aspect, the invention provides a method of activating a bone marrow derived cell or other stem cell in a subject, the method involving contacting a bone marrow cell with an effective amount of an agent having at least two activities that is any one or more of i) inhibition of hsp-90 biological activity; ii) mobilization of a bone marrow derived stem cell; iii) inhibition of apoptosis; and iv) modulation of an immune response

In a related aspect, the invention provides a method of activating a bone marrow derived cell or stem cell in a subject, the method involving contacting a bone marrow cell with an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof, thereby activating the stem cell.

In another aspect, the invention provides a method of increasing sca1⁺, cd45⁺ or cd34⁺ cells in bone marrow or peripheral blood of a subject, the method involving administering an effective amount of an agent having at least two activities that is any one or more of i) inhibition of hsp-90 biological activity; ii) mobilization of a bone marrow derived stem cell; iii) inhibition of apoptosis; and iv) modulation of an immune response.

In yet another aspect, the invention provides a method of activating or mobilizing a bone marrow derived cell in a subject in need thereof, the method involving administering to the subject an effective amount of an agent that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and structural or functional analogs thereof; and administering an effective amount of TE-140 peptide, GM-CSF and/or Stem Cell Factor, where the amount of the agent and GM-CSF and/or Stem Cell Factor is sufficient to activate or mobilize a bone marrow derived cell in the subject.

In another aspect, the invention provides a pharmaceutical composition labeled for the activation or mobilization of a bone marrow derived cell or stem cell, the composition containing an effective amount of an agent having at least two activities that is any one or more of i) inhibition of hsp-90 biological activity; ii) mobilization of a bone marrow derived stem cell; iii) inhibition of apoptosis; and iv) modulation of an immune response.

In another aspect, the invention provides a pharmaceutical composition labeled for the activation or mobilization of a bone marrow derived cell or stem cell, the composition containing triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and functional or structural analogs thereof.

In another aspect, the invention provides a kit for the activation or mobilization of a stem cell, the kit containing an effective amount of an agent having activities that are any one or more of i) inhibition of hsp-90 biological activity; ii) mobilization of a bone marrow derived stem cell; iii) inhibition of apoptosis; and iv) modulation of an immune response. In one embodiment, the agent is any one or more of triptolide, a Tryptigerium derivative of Formula 1-106, oridonin, geldanamycin, celastrol, celastrol methyl ester, dihydrocelastrol diacetate, celastrol butyl ester, dihydrocelastrol, 17-allylamino-17-demethoxygeldanamycin, valproic acid, a combination of TE-140 and celastrol, and instructions for using the kit for the activation or mobilization of a stem cell. In another embodiment, the amount of the agent is sufficient to induce stem cell recruitment to a liver, heart, lung, pancreas, cardiac or other tissue in need of repair or regeneration.

In another aspect, the invention provides a method of inhibiting pancreatic cell death in a subject, the method involving contacting a pancreatic cell at risk of cell death with an agent having at least two activities that is any one or more of i) inhibition of hsp-90 biological activity; ii) mobilization of a bone marrow derived stem cell; iii) inhibition of apoptosis; and iv) modulation of an immune response. In one embodiment, the agent is triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or structural or functional analogs thereof.

In another aspect, the invention provides a method of repairing or regenerating pancreatic tissue in a subject in need thereof, the method involving administering an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide; and recruiting a stem cell to the pancreatic tissue, thereby repairing or regenerating the pancreatic tissue.

In a related aspect, the invention provides a method of treating or preventing diabetes in a subject, the method involving administering an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or structural or functional analogs, thereby treating or preventing diabetes.

In embodiments of the above aspects, the method reduces pancreatic cell death by at least 5%, 10%, 20%, 35%, 50%, 75%, 80%, 90%, or 100% relative to the level in an untreated reference. In other embodiments of the above aspects, the subject has type I or type II diabetes.

In another aspect, the invention provides a method of ameliorating diabetes in a subject in need thereof, the method involving administering to the subject an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide; and administering an effective amount of GM-CSF and/or Stem Cell Factor, thereby ameliorating diabetes.

In various embodiments of the above aspects, the administration repairs or regenerates pancreatic tissue. In other embodiments of the above aspects, the administration inhibits cell death in pancreas. In still other embodiments of the above aspects, the administration increases insulin production.

In another aspect, the invention provides a pharmaceutical composition labeled for the treatment of diabetes, the composition containing an effective amount of a compound that is any one or more of a triptolide, Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof. In one embodiment, the amount is sufficient to recruit a stem cell to a pancreas or is sufficient to inhibit cell death in pancreas or to induce pancreas repair or regeneration.

In yet another aspect, the invention provides a kit for the treatment of diabetes containing an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof, and instructions for using the kit for the treatment of diabetes.

In still another aspect, the invention provides a method of inhibiting liver cell death or damage related to acute liver failure in a subject, the method involving contacting the liver cell with an effective amount of an agent having at least two activities that is any one or more of i) inhibition of hsp-90 biological activity; ii) mobilization of a bone marrow derived stem cell; iii) inhibition of apoptosis; and iv) modulation of an immune response, thereby inhibiting liver cell death or damage. In one embodiment, the agent is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide.

In yet another aspect, the invention provides a method of repairing or regenerating liver tissue in a subject in need thereof, the method involving administering an effective amount of an agent that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs to a subject; and recruiting a stem cell to the liver tissue, thereby repairing or regenerating the liver tissue.

In still another aspect, the invention provides a method of treating or preventing acute liver failure in a subject, the method involving administering an effective amount of an agent that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs, thereby treating or preventing acute liver failure. In one embodiment, the method reduces liver cell death or liver damage by at least 10% relative to the level in an untreated reference. In another embodiment, the subject is identified as negative for hepatitis. In yet another embodiment, the method further involves administering an agent that increases the number, survival, proliferation, or differentiation of a bone marrow derived cell or stem cell. In one embodiment, the agent is TE-140, granulocyte macrophage colony stimulating factor or stem cell factor.

In still another aspect, the invention provides a method of ameliorating acute liver failure in a subject in need thereof, the method involving administering to the subject an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof, where the amount is sufficient to recruit at least one stem cell to a liver; and administering an effective amount of TE-140, GM-CSF and/or Stem Cell Factor, where the amount is sufficient to mobilize a bone marrow derived stem cell in the subject, thereby ameliorating acute liver failure. In one embodiment, the compound is administered locally or systemically. In another embodiment, the compound is administered locally via the hepatic portal vein. In another embodiment, the administration repairs or regenerates liver tissue. In yet another embodiment, the administration inhibits cell death in liver tissue.

In still another aspect, the invention provides a pharmaceutical composition labeled for the treatment of liver failure, the composition containing an effective amount of a compound that is any one or more of a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof. In one embodiment, the amount is sufficient to recruit a stem cell to a liver or inhibit cell death in liver when administered to a subject.

In still another aspect, the invention provides a kit for the treatment of liver failure, containing an effective amount of a compound that is any one or more of a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and instructions for using the kit for the treatment of liver failure.

In yet another aspect, the invention provides a method of inhibiting heart cell death or heart damage in a subject, the method involving contacting a cell with an effective amount of an agent that is any one or more of a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof, thereby inhibiting heart cell death or damage.

In another aspect, the invention provides a method of repairing or regenerating heart tissue in a subject in need thereof, the method involving administering an effective amount of an agent having at least two activities that is any one or more of i) inhibition of hsp-90 biological activity; ii) mobilization of a bone marrow derived stem cell; iii) inhibition of apoptosis; and iv) modulation of an immune response to a subject; and recruiting a stem cell to the heart tissue, thereby repairing or regenerating the heart tissue. In one embodiment, the agent is any one or more of a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide.

In still another aspect, the invention provides a method of treating or preventing heart disease in a subject, the method involving administering an effective amount of a compound that is any one or more of triptolide, a Tryptigerium derivative of Formula 1-106, oridonin, geldanamycin, celastrol, celastrol methyl ester, dihydrocelastrol diacetate, celastrol butyl ester, dihydrocelastrol, 17-allylamino-17-demethoxygeldanamycin, valproic acid, a combination of TE-140 and celastrol, and structural or functional analogs thereof, thereby treating or preventing heart disease. In one embodiment, the method reduces heart cell death or damage by at least 10% relative to the level in an untreated reference. In another embodiment, the subject has coronary heart disease, cardiomyopathy, ischemic heart disease, heart failure, or acute myocardial infarction.

In still another aspect, the invention provides a method of ameliorating heart disease in a subject in need thereof, the method involving administering to the subject an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof, where the amount is sufficient to recruit at least one stem cell to a heart; and administering an effective amount of GM-CSF and/or Stem Cell Factor, where the amount is sufficient to mobilize a bone marrow derived stem cell in the subject, thereby ameliorating heart disease. In one embodiment, the compound is administered systemically or locally via intramyocardial injection. In another embodiment, the administration repairs or regenerates heart tissue or inhibits cell death in heart tissue.

In still another aspect, the invention provides a pharmaceutical composition labeled for the treatment of heart disease, the composition containing an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, a combination of TE-140 and celastrol, and structural or functional analogs thereof. In one embodiment, the amount is sufficient to recruit a stem cell to a heart or inhibit cell death in heart.

In another aspect, the invention provides a kit for the treatment of heart disease, containing an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof, and instructions for using the kit for the treatment of heart disease.

In another aspect, the invention provides a method of repairing or regenerating lung tissue in a subject in need thereof, the method involving administering an effective amount of a compound that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, and structural or functional analogs thereof to a subject; and recruiting a stem cell to the lung tissue, thereby repairing or regenerating the lung tissue.

In yet another aspect, the invention provides a method of treating or preventing lung disease in a subject, the method involving administering to the subject an effective amount of an agent having at least two activities that is any one or more of i) inhibition of hsp-90 biological activity; ii) mobilization of a bone marrow derived stem cell; iii) inhibition of apoptosis; and iv) modulation of an immune response.

In yet another aspect, the invention provides a method of regenerating a hematopoietic system in a subject, the method involving administering to the subject an effective amount of an agent having at least two activities that is any one or more of i) inhibition of hsp-90 biological activity; ii) mobilization of a bone marrow derived stem cell; iii) inhibition of apoptosis; and iv) modulation of an immune response. In one embodiment, the agent is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide.

In still another aspect, the invention provides a method of modulating an immune response in a subject, the method involving administering to the subject an agent that is any one or more of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide. In one embodiment, the method reduces an immune response. In another embodiment, the method prevents or treats diabetes in a subject.

In still another aspect, the invention provides a method of identifying an agent for use in tissue repair or regeneration, the method involving contacting a non-human subject with a test compound and identifying an increase in the number of sca1⁺, cd45⁺ or cd34⁺ cells in the bone marrow or peripheral blood of the rodent.

In various embodiments of any of the above aspects, the method activates a bone marrow derived stem cell of the subject; and recruits the bone marrow derived stem cell to a tissue or organ in need of repair. In other embodiments of any of the above aspects, the method increases cell survival, increases cell proliferation, or reduces cell death in a tissue or organ in need thereof. In various embodiments of any of the above aspects, the method involves administering two or more of the following agents: triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide. In other embodiments of the above aspects, the method involves administering celastrol and triptolide, celastrol and TE-140, or celastrol and geldanamycin. In other embodiments of the above aspects or of any other invention delineated herein, the method further involves identifying a subject as having a disease or disorder characterized by an undesirable increase in cell death or a deficiency in cell number. In one embodiment of a method delineated herein, the method further involves identifying a subject as having a disease or disorder characterized by an undesirable increase in cell death or a deficiency in cell number. In various embodiments of any of the above aspects, the agent is triptolide, a Tryptigerium derivative of Formula 1-106, oridonin, geldanamycin, celastrol, celastrol methyl ester, dihydrocelastrol diacetate, celastrol butyl ester, dihydrocelastrol, 17-allylamino-17-demethoxygeldanamycin, valproic acid, a combination of TE-140 and celastrol, and structural or functional analogs thereof. In other embodiments of the above aspects, the agent increases sca1⁺, cd45⁺ or cd34⁺ stem cells in bone marrow or peripheral blood. For example, the method increases the percentage of sca1⁺, cd45⁺ or cd34⁺ stem cells in bone marrow or peripheral blood by at least about 0.01%, 0.05%, 0.1% or 1% relative to the level of those cells in an untreated reference. In various embodiments of any of the above aspects, the method further involves administering an agent (e.g., granulocyte macrophage colony stimulating factor or stem cell factor) that activates a bone marrow derived cell, or increases the number, survival, proliferation, or differentiation of a bone marrow derived cell. In other embodiments of the above aspects, the method is useful for the treatment or prevention of a disease or disorder characterized by increased cell death or a deficiency in cell number. In other embodiments of the above aspects, the subject is in need of tissue repair or regeneration. In still other embodiments of the above aspects, the tissue in need of repair is bladder, blood system, bone, breast, cartilage, esophagus, fallopian tube, gall bladder, glial cell, heart, intestines, kidney, lung, lymphatic system, muscle, ovaries, pancreas, prostate, spleen, stomach, testes, thymus, thyroid, trachea, urogenital tract, ureter, urethra, uterus, skeletal muscle, and skin. In other embodiments of the above aspects, the method is useful for the treatment or prevention of diabetes, acute liver failure, myocardial infarction, heart failure, cardiomyopathy, lung disease, wounding, hematopoietic cell loss related to radiation or chemotherapeutic ablation, or trauma-induced injury. Preferably, the tissue to be treated is a non-ocular tissue and the disease to be treated is not a protein conformation disorder. In other embodiments of the above aspects, the bone marrow derived cell is a hematopoietic stem cell or a sca1⁺, cd45⁺ and/or cd34⁺ cell. In other embodiments of the above aspects, the agent is administered locally or systemically. In still other embodiments of the above aspects, the cell is in vivo or in vitro. In other embodiments of the above aspects, the method involves locally or systemically administering an isolated stem cell or bone marrow derived cell to the subject. In other embodiments of the above aspects, the cell contains a vector encoding a therapeutic polypeptide.

The invention provides methods of treating or preventing diseases characterized by a decrease in cell number, a decrease in cell function, or an increase in cell death. Other features and advantages of the invention will be apparent from the detailed description, and from the claims.

### Definitions

By “activate” is meant induce to leave a quiescent state. For example, an agent that increases the number of bone marrow derived cells, or their progenitors or progeny in the peripheral blood or increases the number of sca1⁺, cd45⁺ and cd34⁺ cells in bone marrow.

By “agent” is meant any small molecule chemical compound, antibody, nucleic acid molecule, or polypeptide, or fragments thereof.

By “ameliorate” is meant decrease, suppress, attenuate, diminish, arrest, prevent, or stabilize the development or progression of a disease.

By “alteration” is meant a change (increase or decrease) as detected by standard art known methods such as those described herein. As used herein, an alteration includes a 0.01, 0.05, 0.1, 1, 10, 20, 30, 40, 50, 75, 85, or 95% increase or decrease relative to a reference.

By “analog” is meant a molecule that is not identical, but has analogous functional or structural features.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the target of the detection.

By “disease” is meant any condition or disorder that damages or interferes with the normal function of a cell, tissue, or organ.

By “effective amount” is meant the amount of an agent or compound required to ameliorate the symptoms of a disease relative to an untreated patient. The effective amount of active agent or compound(s) used to practice the present invention varies depending upon the manner of administration, the age, body weight, and general health of the subject. Ultimately, the attending physician or veterinarian will decide the appropriate amount and dosage regimen. Such amount is referred to as an “effective” amount. As used herein, an effective amount includes the amount of an agent required to activate a bone marrow derived cell or to recruit a bone marrow derived cell to a tissue or organ. As used herein, an effective amount also includes the amount of an agent required to repair or regenerate a tissue or organ in need thereof, or the amount required to reduce cell death, increase cell survival, or increases cell proliferation.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. Patent law and can mean “ includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “deficiency in cell number” is meant fewer of a specific set of cells than are normally present in a tissue or organ not having a deficiency. For example, a deficiency is a 5%, 10%, 15%, 20%, 30%, 40%, 50%, 60%, 70%, 80%, 90%, or even 100% deficit in the number of cells of a particular cell-type relative to the number of cells present in a naturally-occurring, corresponding tissue or organ. Methods for assaying cell-number are standard in the art, and are described in (Bonifacino et al., Current Protocols in Cell Biology, Loose-leaf, John Wiley and Sons, Inc., San Francisco, Calif., 1999; Robinson et al., Current Protocols in Cytometry Loose-leaf, John Wiley and Sons, Inc., San Francisco, Calif., October 1997). Commercially available kits for determining cell number include CyQUANT Assay for Accurate Cell Quantitation, The CellTiter-Fluor™ Cell Viability Assay(a), and methylene blue assay. Tissue injury, cell death, or a congenital defect can cause a deficiency in cell number.

By “expression vector” is meant a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “hematopoietic stem cell” is meant a bone marrow derived cell capable of giving rise to one or more differentiated cells of the hematopoietic lineage or other differentiated cell types.

By “bone marrow derived cell” is meant a cell or progenitor thereof that arose in the bone marrow.

By “bone marrow derived cell mobilization” is meant increasing the number of bone marrow derived cells available for recruitment to an organ or tissue in need thereof. In particular, increasing the number of sca1+, cd45+ and cd34+ cells in blood.

By “inhibit hsp-90” is meant reduce the chaperone activity of Hsp90 or any other hsp90 biological activity.

By “immune response modifying agent” is meant an agent that stimulates or restores the ability of the immune system to fight disease or that reduces an undesirable immune response.

By “increases or decreases” is meant a positive or negative alteration. Such alterations are by 5%, 10%, 25%, 50%, 75%, 85%, 90% or even by 100% of a reference value.

By “inhibition of apoptosis” is meant to decrease apoptotic cell death. Preferably, the decrease is by at least about 5, 10, 15, 20, 25, 30, % or more.

By “mobilize” is meant move from a resident tissue. For example, a mobilized HSC is one that is moving or has moved from the bone marrow, where the cell typically resides, to the peripheral blood.

By “modulation of an immune response” is meant to desirably alter a disregulated immune response. For example, modulation of an immune response as used herein may refer to a reduction in an autoimmune response or to the normalization of a disregulated immune response.

By “recruit” is meant attract for incorporation into a tissue.

By “regenerating a tissue” is meant replacing cells of a tissue or organ that are missing.

By “repairing tissue damage” is meant ameliorating cell injury, damage, or cell death.

By “risk of cell death” is meant having a propensity to undergo apoptotic, necrotic, or any other form of cell death. Assays for measuring cell death (e.g., necrotic, apoptotic) are known to the skilled artisan. For example, pancreatic, cardiac, or liver cells present in individuals having type I diabetes, cardiac ischemia, or acute liver failure, respectively, are “at risk of cell death.” Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for cell death are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting cell death include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

By “stem cell” is meant a progenitor cell capable of giving rise to one or more differentiated cell types.

By “subject” is meant a mammal, including, but not limited to, a human or non-human mammal, such as a bovine, equine, canine, ovine, rodent, or feline.

By “tissue” is meant a collection of cells having a similar morphology and function. Preferably, a tissue is a non-ocular tissue, such as liver, pancreas, heart, or bone marrow.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition. Typically, an experimental condition is compared with a corresponding untreated control condition.

### BRIEF DESCRIPTION OF THE DRAWINGS

FIGS. 1A-1C are graphs showing the effect of triptolide on sca1⁺, cd45⁺ and cd34⁺ cells in blood.

FIGS. 2A-2C are graphs showing the effect of triptolide on sca1⁺, cd45⁺ and cd34⁺ cells.

FIGS. 3A-3C are graphs showing that celastrol upregulates stem cells present in bone marrow.

FIGS. 4A-4C are graphs showing that celastrol analogs upregulate stem cells present in bone marrow.

FIGS. 5A-5C are graphs showing that celastrol analogs upregulate stem cells present in bone marrow.

FIGS. 6A-6C are graphs showing that celastrol and triptolide in combination upregulate stem cells present in bone marrow.

FIGS. 7A-7C are graphs showing that celastrol and TE-140 peptide in combination upregulate stem cells present in bone marrow.

FIGS. 8A-8V are graphs showing the effect of celastrol and 17-AAG on cytokine production (pg/mL) in mouse blood pre- and post-treatment periods. In each graph, time post-treatment (in hours) is shown on the X-axis and cytokine concentration (pg/ml) is shown on the Y-axis.

FIG. 9 is a graph showing the effect of celastrol on blood glucose levels in a mouse model of diabetes.

FIG. 10 is a graph showing the effect of celastrol on blood glucose levels in a mouse model of diabetes following glucose challenge test.

FIGS. 11A-11D depict the histological examination of mouse liver with acute liver failure (ALF) induced by thioacetamide (TAA) and the effect of celastrol on liver tissue with ALF. In FIGS. 11A-11D, PT denotes the portal triad, and CV denotes the central vein. FIG. 6A depicts a representative histological section from normal mouse liver (200× magnification). FIG. 11B depicts a representative histological section of mouse liver parenchyma 24 hours after ALF induction by lethal dose of TAA (1000 mg/kg) (200× magnification). Black arrowheads denote hemorrhaging and necrotic liver parenchyma. A white arrowhead denotes normal undamaged hepatocytes. FIG. 11C depicts a representative histological section of mouse liver parenchyma 3 days after ALF induction by TAA (500 mg/kg) (100× magnification). FIG. 11D depicts a representative histological section of mouse liver parenchyma 3 days after ALF induction by TAA (500 mg/kg) and subsequent administration of celastrol (3 mg/kg) (200× magnification). This analysis shows that celastrol rescued liver tissue in mice with ALF induced by TAA.

FIG. 12 depicts the survival of C57BL6/J mice with acute liver failure (ALF) induced by thioacetamide (TAA) and the effect of celastrol treatment on ALF survival. Mice were administered either a Placebo, TAA (1000 mg/kg), TAA (1000 mg/kg) followed by celastrol (3 mg/kg) (TAA+C). This analysis shows that celastrol rescued liver tissue in mice with ALF induced by TAA.

FIG. 13 depicts the effects of celastrol treatment in C57BL6/J mice with heart disease induced by doxorubicin (DOX). Mice were administered either DOX (20 mg/kg) and placebo (D+P) or DOX followed by celastrol (3 mg/kg) (D+C). FIG. 13 depicts survival in mice with DOX induced heart disease and the effect of celastrol treatment on survival from heart disease. This analysis shows that celastrol rescued heart tissue in mice with DOX induced heart disease.

FIGS. 14A and 14B show that oridonin activates stem cell populations in bone marrow and mobilizes them into peripheral blood in C57BL6/J mice.

FIGS. 15A and 15B show that valproic acid activates stem cell populations in bone marrow and mobilizes them into peripheral blood in C57BL6/J mice.

FIG. 16 is a graph showing that celastrol induces normoglycemia in NOD mice, which are a recognized mouse model of diabetes.

FIG. 17 is a graph showing that celastrol modulated the disregulated immune response causing diabetes in the NOD mice.

### DETAILED DESCRIPTION OF THE INVENTION

The present invention generally provides therapeutic and prophylactic compositions and methods, and their use in activating stem cells in bone marrow for the repair or regeneration of tissues and organs. The invention is based, at least in part, on the discovery that agents described herein activated bone marrow derived stem cells and were useful for the treatment of diseases characterized by a cellular deficiency, such as liver failure, diabetes, and cardiomyopathy. In particular, agents of the invention activated CD34-expressing cells, CD45-expressing cells, and Sca-1 expressing cells in bone marrow. Following activation, cells moved from the bone marrow into the peripheral blood in a time dependent manner. Treatment with celastrol increased pancreatic function in a mouse model of diabetes, and markedly increased survival in mouse models of acute liver failure and heart failure.

These surprising discoveries indicate that agents of the invention (e.g., triptolide, a Tryptigerium derivative of Formula 1-106, oridonin, geldanamycin, celastrol, celastrol and celastrol analogs, including dihydrocelastrol, dihydrocelastrol diacetate, and pristimerol, and the geldanamycin analog 17-AAG, oridonin, valproic acid, and a combination of TE-140 peptide (e.g., 4F-benzoyl-TN14003) and celastrol and analogs thereof) are useful for the repair or regeneration of a variety of tissues, including but not limited to, liver, lung, heart, and pancreas, as well as for the regeneration or repair of a damaged hematopoietic system (e.g., for repairing hematopoietic cell loss related to radiation or chemotherapeutic ablation). Accordingly, the invention provides agents for use in the repair or regeneration of a tissue or organ in need thereof, as well as therapeutic combinations that include any one or more agents described herein. In particular embodiments, a therapeutic combination of the invention contains celastrol and celastrol derivatives in combination with geldanamycin and geldanamycin analogs (e.g., 17-AAG); celastrol and celastrol derivatives in combination with triptolide; and celastrol and celastrol derivatives in combination with a TE-140 peptide having an amino acid sequence described herein.

### Therapeutic Agents

The invention provides therapeutic agents useful for the activation of a bone marrow stem cell or for the repair or regeneration of a tissue or organ. Agents useful in the methods of the invention include, but are not limited to, triptolide, a Tryptigerium derivative of Formula 1-106, oridonin, geldanamycin, celastrol, celastrol and celastrol analogs, including dihydrocelastrol, dihydrocelastrol diacetate, and pristimerol, and the geldanamycin analog 17-AAG, oridonin, valproic acid, celastrol and celastrol derivatives in combination with geldanamycin and geldanamycin analogs (e.g., 17-AAG); celastrol and celastrol derivatives in combination with triptolide; and celastrol and celastrol derivatives in combination with a TE-140 peptide having an amino acid sequence described herein, or any other agent delineated herein or an analog thereof. Other agents useful in the methods of the invention include those having one or more of the following biological activities: bone marrow stem cell activation, HSP-90 inhibition, apoptosis modulation, and/or immunomodulatory activity. Agents having one, two, three, four of such activities are useful for the repair or regeneration of a tissue or organ. In one embodiment, an agent of the invention has all of these activities. If desired, any of the compounds described herein may be used alone or in any combination of between 1 and 115 compounds. Preferably, therapeutic agents are used in combination of 1, 2, 3, 4, 5 or more.

Celastrol, a quinone methide triterpene derived from the medicinal plant Tripterygium wilfordii, has been used to treat chronic inflammatory diseases. Tripterygium wilfordii has a long history in Chinese herbal medicine (Li et al., Anti-Inflam. Components of Tript. Wilfordii Hook F. (1993) Int. J. Immunotherapy IX(3): 181-187) for the treatment of fever, chills, edema and inflammation. In China, celastrol has been administered as a refined extract that contains predominantly triterpenes. Celastrol analogs and derivatives include, but are not limited to, celastrol methyl ester, dihydrocelastrol diacetate, pristimerol, celastrol butyl ester, dihydrocelastrol, and salts or structural or functional analogs thereof. Such compounds may be used in the compositions and methods of the invention.

Other compositions useful in the methods of the invention are triterpenoids derived from Tripterygium wilfordii. Such compounds include triptodiolide, triptonide, triptonoterpenol, triptophenolide, and triptophenolide methyl ether. Methods for extracting therapeutic agents from Tripterygium wilfordii are described, for example, in U.S. Pat. No. 4,005,108, which is incorporated by reference in its entirety. Other triterpine constituents are likely to be useful in the methods of the invention individually or in combination with celastrol or any other agent described herein. One preferred combination is celastrol and triptolide. Triptolide is a biologically active diterpene isolated from Tripterygium that is a potent inhibitor of NF-κB- and NF-AT-mediated transcription. Other derivatives of Tripterygium useful in the methods of the invention have formulas 1-106.

Other compounds useful alone or in combination with celastrol or celastrol analogs, include the benzoquinone ansamycin antibiotic geldanamycin, as well as its derivative 17-allylamino-17-demethoxygeldanamycin (17-AAG). Other compounds useful alone or in combination with celastrol and/or geldanamycin in the methods of the invention are radicicol, novobiocin, EC102, radicicol, geranylgeranylacetone, paeoniflorin, PU-DZ8, H-71, TE-140 peptide (e.g., 4F-benzoyl-TN14003) and celastrol, as well as combinations of these agents. In particular, a therapeutic combination of the invention contains celastrol and celastrol derivatives in combination with geldanamycin and geldanamycin analogs (e.g., 17-AAG); celastrol and celastrol derivatives in combination with triptolide; and celastrol and celastrol derivatives in combination with a TE-140 peptide.

In one embodiment, dosages of celastrol range from at least about 0.001 mg/kg to about 6 mg/kg, from about 0.002 mg/kg to about 3 mg/kg, about 0.002 mg/kg to about 2 mg/kg, or from about 0.002 to about 1.5 mg/kg. In particular, the bottom of the range may be any number between 0.001 and 5 mg/kg, and the top of the range may be any number between 0.002 and 6 mg/kg. In other embodiments, at least about 10 mg, 20 mg, 30 mg, 40 mg, or 50 mg of celastrol is administered to a subject per day.

Dosages of geldanamycin or 17-AAG range from least about 0.001 mg/kg to about 600 mg/kg, from about 1 mg/kg to about 300 mg/kg, or from about 10 mg/kg to about 200 mg/kg. In one embodiment, geldanamycin or 17-AAG is provided intravenously at 60 mg/kg or 160 mg/kg. In particular, the bottom of the range may be any number between 0.001 and 600 mg/kg, and the top of the range may be any number between 0.002 and 599 mg/kg.

Triptolide dosages or dosages of triptolide analogs vary from at least about 1 to 1000 μg/kg body weight, or from about 1 μg to about 500 μg/kg body weight, or from about 50 μg to about 100 μg/kg body weight. In other embodiments, less than about 1 mg, 2 mg, 3 mg, 4 mg, 5 mg, 6 mg, 7 mg, or 8 mg/day of triptolide or a triptolide derivative is administered per day.

Oridonin dosages vary from about 1-100 mg/day. For example, 5, 10, 20, 30, 40, or 50 mg/day is administered. In other embodiments, 10, 15, 20, or 30 mg/day is administered. In still other embodiments, 15, 20, 25, 30 mg/day is administered.

Valproic acid dosages range from 1-50 mg/day. In particular embodiments, valproic acid is administered at 1, 5, 10, 15, 20, or 30 mg at least once, twice, or three times per day. In a specific embodiment, valproic acid is administered at 15 mg, three times per day.

### Therapeutic Methods

The invention provides compositions and methods that activate and/or mobilize sca1⁺, cd45⁺ and/or cd34⁺ stem cells in bone marrow, that recruit stem cells to a tissue or organ of interest, that reduce excess cell death, that increase cell proliferation, or that otherwise treat a disease or disorder characterized by a deficiency in cell number or cell function. As described herein, compositions comprising triptolide, a Tryptigerium derivative of Formula 1-106, oridonin, celastrol, celastrol and celastrol analogs (e.g., dihydrocelastrol, dihydrocelastrol diacetate, and pristimerol), geldanamycin and geldanamycin analogs (e.g., 17-AAG), oridonin, valproic acid, and a combination of TE-140 peptide (e.g., 4F-benzoyl-TN14003) and celastrol and analogs thereof, alone or in combination, are useful for activating stem cells, for recruiting stem cells to a tissue in need of repair or regeneration, for reducing cell death in a cell, for increasing cell growth or proliferation, or otherwise treating a disease or disorder characterized by a deficiency in cell number or a deficiency in the biological activity of a tissue or organ. In one embodiment, methods of the invention increase the availability of cells that are useful for the repair or regeneration of a damaged tissue. Such cells may be generated by the activation of a stem cell (e.g., a bone marrow derived stem cell, such as a hematopoietic stem cell). Alternatively, cells useful for repair or regeneration are recruited to a tissue of interest where they repair or regenerate the tissue, or increase the biological activity of the tissue. In one embodiment, the recruited cells engraft and differentiate to generate a cell type of interest (e.g., a bone marrow stem cell or HSC gives rise to an alternate cell type, such as a liver cell, heart cell, or pancreatic cell). In an alternate approach, the methods of the invention reduce undesirable cell death in a tissue or organ.

The invention provides for the treatment of diseases and disorders associated with a deficiency in cell number (e.g., a reduction in the number of pancreatic, hepatic, lung, or cardiac cells), an excess in undesirable cell death (e.g., necrotic or apoptotic cell death), or an insufficient level of cell biological activity (e.g., a deficiency in insulin production, reduction in liver function, reduction in lung function, reduction in cardiac function). In one embodiment, the invention provides compositions for the treatment of diabetic subjects, including subjects having type I or type II diabetes. In particular, the invention provides compositions and methods useful for the treatment of subjects who lack sufficient levels of insulin due to a decrease in the number or activity of insulin producing pancreatic cells. In other embodiments, the invention provides compositions useful for the treatment of acute liver failure, hepatitis, cirrhosis, or any other disease or disorder that damages the liver.

Many diseases associated with a deficiency in cell number are characterized by an increase in cell death. Such diseases include, but are not limited to, acute liver failure, heart failure, chronic obstructive pulmonary disease, neurodegenerative disorders, stroke, myocardial infarction, or ischemic injury. In one embodiment, the invention provides compositions and methods for the treatment of subjects having an increase in cardiac cell death or a decrease in cardiac function related to myocardial infarction, heart disease, cardiomyopathy, or heart failure. Injuries associated with trauma can also result in a deficiency in cell number in the area sustaining the wound. Thus, the invention provides compositions and methods useful in promoting wound healing.

Expressly excluded from the methods of the invention are the treatment of an ocular tissue, an ocular disease, and the treatment of protein conformation diseases (PCD). By “protein conformational disease” is meant a disease or disorder whose pathology is related to the presence of a misfolded protein. For example, a protein conformational disease is caused when a misfolded protein interferes with the normal biological activity of a cell, tissue, or organ.

The present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a disease or disorder or symptom thereof characterized by a deficiency in cell number or an undesirable increase in cell death. The method includes the step of administering to the mammal a therapeutic amount of an amount of a composition of the invention sufficient to treat the disease or disorder or symptom thereof, under conditions such that the disease or disorder is treated.

In one embodiment, an agent of the invention contacts a cell, tissue, or organ (e.g., liver, heart, pancreas, bladder, brain, spinal neuron, motor neuron, glial cell, esophagus, fallopian tube, heart, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage) in vivo or in vitro to reduce cell death, to increase cell survival, proliferation, or to otherwise repair or regenerate the cell, tissue, or organ (e.g., by recruiting stem cells or reducing cell death). Alternatively, activated bone marrow derived stem cells are locally or systemically administered to a subject prior to, concurrent with, or subsequent to administration of a compound of the invention to enhance stem cell recruitment and ameliorate the disease, disorder, or injury. In one embodiment, compositions and methods of the invention ameliorate a disease, disorder, or injury characterized by a deficiency in cell number or function in the affected organ. In various embodiments, agents of the invention are administered systemically, or by local injection to a site of disease or injury, by sustained infusion, or by micro-injection under surgical conditions (Wolff et al., Science 247:1465, 1990).

In preferred embodiments, a composition or method of the invention increases the biological activity of a pancreatic, hepatic, or cardiac cell, tissue or organ by at least 5%, 10%, 20%, 30%, 40%, 50%, 60%, 70%, 80%, 90%, 100%, 150%, or even by as much as 200%, 300%, 400%, or 500% compared to a corresponding, naturally-occurring tissue or organ. In various embodiments, a composition or method of the invention increase the biological function of a diseased or damaged tissue by at least about 5%, 10%, 25%, 50%, 75%, 100%, 200%, or even by as much as 300%, 400%, or 500% relative to a corresponding untreated control. Biological functions of the tissue or organ amenable to assay include enzyme production, excretion of waste, secretion, electrical activity, hormone production, or other metabolic activity. For example, liver function is assayed using liver function tests or a liver panel that measures liver enzyme levels, bilirubin levels, and albumin levels. Biological functions of a pancreatic tissue or organ amenable to assay include insulin production. Methods for assaying insulin production include measuring blood glucose levels. Any number of standard methods are available for assaying cardiovascular function.

Preferably, cardiovascular function in a subject (e.g., a human) is assessed using non-invasive means, such as measuring net cardiac ejection (ejection fraction, fractional shortening, and ventricular end-systolic volume) by an imaging method such echocardiography, nuclear or radiocontrast ventriculography, or magnetic resonance imaging, and systolic tissue velocity as measured by tissue Doppler imaging. Systolic contractility can also be measured non-invasively using blood pressure measurements combined with assessment of heart outflow (to assess power), or with volumes (to assess peak muscle stiffening). Measures of cardiovascular diastolic function include ventricular compliance, which is typically measured by the simultaneous measurement of pressure and volume, early diastolic left ventricular filling rate and relaxation rate (can be assessed from echoDoppler measurements). Other measures of cardiac function include myocardial contractility, resting stroke volume, resting heart rate, resting cardiac index (cardiac output per unit of time [L/minute], measured while seated and divided by body surface area [m²])) total aerobic capacity, cardiovascular performance during exercise, peak exercise capacity, peak oxygen (O₂) consumption, or by any other method known in the art or described herein.

Other methods for assaying the biological function of an organ, such as the pancreas, liver, lung, or heart are routine, and are known to the skilled artisan (e.g., Guyton et al., Textbook of Medical Physiology, Tenth edition, W.B. Saunders Co., 2000).

Methods of the invention are useful for treating or stabilizing in a subject (e.g., a human or mammal) diabetes, heart failure, acute liver failure, chronic obstructive pulmonary disease, or another condition, disease, or disorder characterized by increased cell death, reduced cell function, or reduced cell number.

In other preferred embodiments, the method increases the number of cells of the tissue or organ by at least 5%, 10%, 20%, more desirably by at least 25%, 30%, 35%, 40%, 50%, 60%, or even by as much as 70%, 80%, 90 or 100% compared to a corresponding control tissue or organ. Methods for assaying cell number, survival or proliferation are known to the skilled artisan and are described, for example, by Bonifacino et al., (Current Protocols in Cell Biology Loose-leaf, John Wiley and Sons, Inc., San Francisco, Calif.).

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compositions herein may be also used in the treatment of any other disorders in which an increase in the number of sca1⁺, cd45⁺ and/or cd34⁺ stem cells in bone marrow or peripheral blood is desired. The compositions herein may be also used in the treatment of any other disorders in which a deficiency in cell number may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a deficiency in cell number, in which the subject has been administered a therapeutic amount of a compound herein sufficient to treat the disease or symptoms thereof. The level of Marker determined in the method can be compared to known levels of Marker in either healthy normal controls or in other afflicted subjects to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Hematopoietic Stem Cells

As reported herein, agents of the invention (e.g., triptolide, geldanamycin, oridonin, celastrol, dihydrocelastrol, dihydrocelastrol diacetate, pristimerol, celastrol and celastrol derivatives in combination with geldanamycin and geldanamycin analogs (e.g., 17-AAG); celastrol and celastrol derivatives in combination with triptolide; and celastrol and celastrol derivatives in combination with a TE-140 peptide having an amino acid sequence described herein, such as the TE-140 peptide (H-Arg-Arg-Nal-Cys-Tyr-Arg-Lys-dLys-Pro-Tyr-Arg-Cit-Cys-Arg-OH)) are useful for activating bone marrow derived stem cells. For example, as reported in more detail below, each of triptolide, geldanamycin, oridonin, celastrol, dihydrocelastrol, dihydrocelastrol diacetate, pristimerol, a combination of celastrol and triptolide, and a combination of TE-140 peptide and celastrol activated bone marrow derived stem cells (e.g., increased the percentage of sca1⁺, cd45⁺ and/or cd34⁺ stem cells). For example, triptolide and oridonin each increased the percentage of sca1⁺, cd45⁺ and/or cd34⁺ stem cells in peripheral blood. Without wishing to be bound by theory, triptolide, 17-AAG, oridonin, celastrol, and a combination of TE-140 peptide (e.g., 4F-benzoyl-TN14003) and celastrol, or functional or structural analogs thereof, induce bone marrow stem cell activation or mobilization. Hematopoietic stem cells are bone marrow-derived cells that represent an endogenous source known for their reparative potential as well as for their plasticity. Celastrol also reduced tissue damage in mouse models of diabetes, acute liver failure, and heart disease. Other agents having similar biological activities are expected to be equally useful in tissue repair or regeneration. In particular, agents having one or more of the following activities: bone marrow stem cell activation, HSP-90 inhibition, apoptosis modulation, and/or immunomodulatory activity, are useful for the repair or regeneration of a tissue. Also useful in the methods of the invention are agents that increase the percentage of sca1⁺, cd45⁺ and/or cd34⁺ stem cells in bone marrow or in peripheral blood.

Without wishing to be bound by theory, it is possible that activated bone marrow-derived stem cells are recruited to areas of injury to effect the repair or regeneration of a diseased or injured cell, tissue, or organ. In one embodiment, stem cells are recruited to the pancreas, heart, lung, or liver to affect the repair or regeneration of the pancreas, heart, lung, or liver. If desired, the number of hematopoietic stem cells present in the circulation of a subject may be increased prior to, during, or following treatment of a subject with an agent of the invention. In one embodiment, this increase in hematopoietic stem cell number is accomplished by mobilizing hematopoietic stem cells present in the bone marrow of the subject by administering one or more of granulocyte-macrophage colony stimulating factor (G-CSF), stem cell factor (SCF), IL-8, SDF-1 (stromal derived factor), interleukin-1 (IL-1), interleukin-3 (IL-3), interleukin-6 (IL-6), interleukin-7 (IL-7), interleukin-8 (IL-8), interleukin-11 (IL-11), interleukin-12 (IL-12), and NIP-1α, stem cell factor (SCF), fims-like tyrosine kinase-3 (flt-3), transforming growth factor-β (TGF-β), an early acting hematopoietic factor, described, for example in WO 91/05795, and thrombopoietin (Tpo), FLK-2 ligand, FLT-2 ligand, Epo, Oncostatin M, and MCSF, in combination with an agent of the invention.

SDF-1 is a potent cytokine that induces the recruitment of stem cells. Administration of G-CSF and/or SDF-1 is expected to increase the number of HSC in the peripheral blood and to enhance subsequent HSC recruitment to a damaged or diseased tissue or organ. Preferably, hematopoietic stem cells of the invention fail to express or express reduced levels of any one or more of the following markers: Lin⁻, CD2⁻, CD3⁻, CD7⁻, CD8⁻, CD10⁻, CD14⁻, CD15⁻, CD16⁻, CD19⁻, CD20⁻, CD33⁻, CD38⁻, CD71⁻, HLA-DR⁻, and glycophorin A⁻. In other embodiments, triptolide, celastrol, oridonin, geldanamycin, or other compounds of the invention activate a stem cell that resides in bone marrow or blood.

In other preferred embodiments, the method increases the number of sca1⁺, cd45⁺ and/or cd34⁺ stem cells in bone marrow or in peripheral blood by at least about 0.01%, 0.02%, 0.03%, 0.04%, 0.05%, 1%, 3%, 5%, 10%, 20%, 30% or more relative to the number of cells present prior to treatment. Methods for assaying HSC cell surface markers are known to the skilled artisan.

In one embodiment, administration of an effective amount of an agent described herein is sufficient to increase the number of cells in a treated tissue relative to the tissue prior to treatment or relative to an untreated control tissue. Alternatively, administration of an effective amount of an agent described is sufficient to reduce cell death or increase cell survival or proliferation by at least about 1%, 3%, 5%, 10%, 25%, 50%, 75% or more relative to an untreated control tissue. In other embodiments, administration of an effective amount of an agent described is sufficient to recruit one or more bone marrow derived stem cells to the tissue or organ. While the particular level of stem cell recruitment will vary depending on the therapeutic objective to be achieved, desirably at least about 1,2, 3, 5, 10, 25, 100, 500, 1000, 2500, 5000 stem cells are recruited. In other embodiments, at least about 0.1, 0.5, 1, 2, 5, 10, or 15% of the cells present in the tissue of interest are recruited stem cells after treatment. In other embodiments, at least 25%, 35%, or 50% of cells are recruited stem cells.

In various embodiments, agents of the invention are administered by local injection to a site of disease or injury, by sustained infusion, or by micro-injection under surgical conditions (Wolff et al., Science 247:1465, 1990). In other embodiments, the agents are administered systemically to a bone marrow tissue of a subject having a deficiency in cell number that can be ameliorated by cell regeneration. In yet other embodiments, the agents are administered systemically to a tissue or organ of a subject having a deficiency in cell number that can be ameliorated by cell regeneration.

### Pharmaceutical Compositions

The present invention features pharmaceutical preparations comprising agents useful for the repair or regeneration of tissues or organs. In particular, the invention provides pharmaceutical compositions comprising triptolide, a Tryptigerium derivative of Formula 1-106, oridonin, geldanamycin, celastrol, celastrol and celastrol analogs, including dihydrocelastrol, dihydrocelastrol diacetate, and pristimerol, and the geldanamycin analog 17-AAG, oridonin, valproic acid, celastrol and celastrol derivatives in combination with geldanamycin and geldanamycin analogs (e.g., 17-AAG); celastrol and celastrol derivatives in combination with triptolide; and celastrol and celastrol derivatives in combination with a TE-140 peptide having an amino acid sequence described herein.

In other embodiments, the invention provides agents having any one or more of the following biological activities: bone marrow stem cell activation, HSP-90 inhibition, apoptosis modulation, and/or immunomodulatory activity. Preferably, the agents have at least two or three of the aforementioned activities. In one embodiment, the agents activate a bone marrow stem cell and reduce apoptosis. Compositions of the invention may be used alone or in any combination. Preparations comprising such compounds have both therapeutic and prophylactic applications. Compounds useful in the methods described herein include those that activate and/or mobilize sca1⁺, cd45⁺ and/or cd34⁺ stem cells in bone marrow and blood; compounds that ameliorate diabetes, acute liver failure, chronic obstructive pulmonary disease, and/or heart failure, as well as other indications characterized by increased cell death, or reduced cell function, including but not limited to, diseases and disorders affecting liver, heart, bladder, brain, spinal neuron, motor neuron, glial cell, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage.

Compounds of the invention may be administered as part of a pharmaceutical composition. The compositions should be sterile and contain a therapeutically effective amount of the agents of the invention in a unit of weight or volume suitable for administration to a subject. The compositions and combinations of the invention can be part of a pharmaceutical pack, where each of the compounds is present in individual dosage amounts.

Pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution.

The compounds may be combined, optionally, with a pharmaceutically acceptable excipient. The term “pharmaceutically-acceptable excipient” as used herein means one or more compatible solid or liquid filler, diluents or encapsulating substances that are suitable for administration into a human. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be also included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject in need of an increase in the number or mobilization of sca1⁺, cd45⁺ and/or cd34⁺ stem cells, an effective amount is sufficient to increase, activate, or mobilize the percentage of the total population of such cells by at least about 0.01%, 0.02%, 0.05%, 0.06%, 0.1%, 0.2% or even by as much as 0.3%. With respect to a subject having a disease or disorder characterized by a reduction in organ function or a deficiency in cell number, an effective amount is sufficient to attract at least one stem cell to the tissue; or sufficient to stabilize, slow, or reduce a symptom associated with a pathology. Generally, doses of the compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that subject tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. In one embodiment, the intravenous administration is by the hepatic portal vein. In another embodiment, the intramuscular administration is by intramycardial injection. Oral administration can be preferred for prophylactic treatment because of the convenience to the subject as well as the dosing schedule.

Pharmaceutical compositions of the invention can optionally further contain one or more additional proteins as desired. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian protein which has been introduced according to standard recombinant DNA techniques; or from the human biological fluids (e.g., blood, milk, lymph, urine or the like) or from transgenic animals that contain a gene that expresses a human protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising a compound of the present invention can contain multivalent metal ions, such as calcium ions, magnesium ions and/or manganese ions. Any multivalent metal ion that helps stabilizes the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly(2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2, 3 dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Those of skill in the art will recognize that the best treatment regimens for using compounds of the present invention to activate or mobilize bone marrow derived stem cells, to treat a pancreatic disease (e.g., type I or type II diabetes), a liver disease, a lung disease, a heart disease, or any other disease or disorder characterized by a reduction in cell number or cell function, or an increase in cell death, can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular subject.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular subject.

### Methods of Delivery

An agent of the invention may be administered by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. In one embodiment, a therapeutic composition of the invention is provided via an osmotic pump. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active polypeptide therapeutic(s), the composition may include suitable parenterally acceptable carriers and/or excipients. The active polypeptide therapeutic (s) may be incorporated into an osmotic pump, microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active fusion polypeptide therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

In one embodiment, a therapeutic composition of the invention (e.g.,) is provided locally via a canula. For example, for reprogramming a liver-derived cell to an insulin producing cell a composition of the invention is provided to the liver via the portal vein. More preferably, the composition is directed specifically to a single lobe of the liver by providing the composition (e.g., via a canula) to only one of the three branches of the portal vein, such that only one lobe of the liver comprises insulin producing cells. In other embodiments, a composition of the invention is provided via an osmotic pump. Desirably, the osmotic pump provides for the controlled release of the composition over 1-3 days, 3-5 days, 5-7 days, or for 2, 3, 4, or 5 weeks.

### Screening Assays

As discussed herein, compounds that are useful for the repair or regeneration of a tissue or organ can be identified according to any method delineated herein. In particular, agents are screened for those that inhibit hsp-90 biological activity, mobilize a bone marrow derived stem cell, inhibit apoptosis, and modulate an immune response. Agents having at least two of these biological activities are identified as useful in the methods of the invention.

Other screening methods are useful to identify agents that activate and/or mobilize sca1⁺, cd45⁺ and/or cd34⁺ stem cells in bone marrow. Agents identified as activating such stem cells are also identified as useful in the methods of the invention. Any number of methods are available for carrying out screening assays to identify such compounds. In one approach, compounds are screened to identify those that increase the number of sca1⁺, cd45⁺ and/or cd34⁺ cells present in bone marrow or peripheral blood. Compounds that increase the number of such cells are useful in the methods of the invention. In other embodiments, the survival or proliferation of such cells is increased. If desired, the efficacy of an identified compound is assayed in an animal model having a disease (e.g., an animal model of liver failure, diabetes, chronic obstructive pulmonary disease, or cardiac cell death).

As discussed herein, compounds that reduce cell death in a tissue or organ of interest (e.g. pancreas, liver, heart, etc.), increase cell function in a tissue or organ of interest (e.g. pancreas, liver, heart, etc.), increase the repair or increase regeneration of a tissue of interest (e.g. pancreas, liver, heart, etc.), or induce stem cell recruitment to a tissue of interest (e.g. pancreas, liver, heart, etc.) having a deficiency in cell number are useful in the methods of the invention. Any number of methods are available for carrying out screening assays to identify such compounds.

In one approach, compounds are screened to identify those that reduce apoptotic or necrotic cell death in a tissue or organ of interest (e.g. pancreas, liver, heart, etc.). If desired, the efficacy of the identified compound is assayed in an animal model having a disease (e.g., an animal model of having a deficiency in cell number caused, for example, by cell death). In one embodiment, a pancreatic cell is contacted with a test compound prior to, during or following treatment with streptozotocin to induce pancreatic cell death. In another embodiment, a liver cell is contacted with a test compound prior to, during or following treatment with thiomacetamide to induce liver cell death. In another embodiment, a cardiac cell is contacted with a test compound prior to, during or following treatment with doxorubicin to induce cardiac cell death. Compounds that reduce cell death (e.g., by at least about 5%, 10%, 25%, 50%, 75%, or most preferably by at least 100%) in a tissue or organ of interest (e.g. pancreas, liver, heart, etc.) are identified as useful for the treatment of a pathology in a tissue or organ of interest (e.g. pancreas, liver, heart, etc.). If desired, the biological function of the cell, tissue or organ is assayed using any method known in the art or described herein. Compounds that increase the biological function are identified as useful in the methods of the invention.

Alternatively, compounds are screened to identify those that increase stem cell recruitment to a tissue or organ of interest (e.g. pancreas, liver, heart, etc.). In one embodiment, stem cell recruitment is assayed in a chimeric mouse injected locally or systemically with GFP⁺ expressing stem cells. The presence of GFP⁺ cells is assayed, for example, by examining tissue sections in an organ of interest (e.g. pancreas, liver, heart, etc.) using fluorescence microscopy. In other embodiments, the survival or differentiation of such cells is assayed using cell specific markers. Compounds that increase stem cell recruitment (e.g., by at least about 5%, 10%, 25%, 50%, 75%, or most preferably by at least 100%) in a tissue or organ of interest (e.g. pancreas, liver, heart, etc.) are identified as useful for the treatment of a pathology in a tissue or organ of interest (e.g. pancreas, liver, heart, etc.).

If desired, the efficacy of the identified compound is assayed in an animal model having a disease (e.g., an animal model of having a deficiency in cell number caused, for example, cell death). In one embodiment, a pancreatic cell is contacted with a test compound prior to, during or following treatment with streptozotocin to induce pancreatic cell death. In another embodiment, a liver cell is contacted with a test compound prior to, during or following treatment with thiomacetamide to induce liver cell death. In another embodiment, a cardiac cell is contacted with a test compound prior to, during or following treatment with doxorubicin to induce cardiac cell death.

### Test Compounds and Extracts

In general, compounds useful in methods of the invention are identified from large libraries of either natural product or synthetic (or semi-synthetic) extracts or chemical libraries according to methods known in the art. Similarly, methods known in the art for screening libraries can be used to identify compounds capable of reducing pancreatic, cardiac, or hepatic cell death, increasing pancreatic, cardiac, or hepatic cell function, or increasing the repair or regeneration of pancreatic, cardiac, or hepatic tissue. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity (i.e., in activating, mobilizing or recruiting stem cells to a tissue of interest; reducing pancreatic, cardiac, or hepatic cell death or increasing pancreatic, cardiac, or hepatic function) should be employed whenever possible.

When a crude extract is found to have one or more of the following activities: to activate, mobilize or recruit sca1⁺, cd45⁺ and/or cd34⁺ stem cells to a tissue of interest; to reduce cell death in a tissue or organ of interest (e.g., pancreas, liver, heart, or other tissue or organ); recruit stem cells to a tissue or organ of interest; or otherwise induce repair or regeneration in a tissue or organ of interest, further fractionation of the positive lead extract is necessary to isolate chemical constituents responsible for the observed effect. Thus, the goal of the extraction, fractionation, and purification process is the careful characterization and identification of a chemical entity within the crude extract that has the activity. Methods of fractionation and purification of such heterogenous extracts are known in the art. If desired, compounds shown to be useful agents for the treatment of any pathology related to a disease requiring the repair or regeneration of a tissue of interest are chemically modified according to methods known in the art.

Methods for Activating Stem Cells and/or Increasing Stem Cell Recruitment to a Tissue

Administration of an agent of the invention is useful to activate and/or mobilize sca1⁺, cd45⁺ and/or cd34⁺ stem cells in bone marrow. Alternatively or in addition, administration of an agent of the invention is useful for the treatment or prevention of a disease characterized by a deficiency in cell number (e.g., diabetes, liver failure, lung disease, chronic obstructive pulmonary disease, heart disease, etc.). Without wishing to be tied to theory, it is likely that agents of the invention recruit stem cells (e.g., bone marrow derived stem cells) to a tissue (e.g., pancreatic tissue, liver tissue, heart tissue, etc.), where they ameliorate a disease or disorder. Alternatively, agents of the invention repair a tissue or organ by reducing cell death, increasing cell survival, or increasing cell proliferation. If desired, agents of the invention are administered in combination with isolated stem cells. Preferably, the administered stem cells are from the same subject.

Methods of isolating hematopoietic stem cells are known in the art. In one embodiment, hematopoietic stem cells are isolated from the blood using apheresis. Apheresis for total white cells begins when the total white cell count is about 500-2000 cells/μl and the platelet count is about 50,000/μl. Daily leukapheris samples may be monitored for the presence of CD34⁺ and/or Thy-1⁺ cells to determine the peak of stem cell mobilization and, hence, the optimal time for harvesting peripheral blood stem cells. Various techniques may be employed to separate the cells by initially removing cells of dedicated lineage (“lineage-committed” cells), if desired. Monoclonal antibodies are particularly useful for identifying markers associated with particular cell lineages and/or stages of differentiation. The antibodies may be attached to a solid support to allow for crude separation. The separation techniques employed should maximize the viability of the fraction to be collected.

The use of separation techniques include those based on differences in physical properties (e.g., density gradient centrifugation and counter-flow centrifugal elutriation), cell surface properties (lectin and antibody affinity), and vital staining properties (mitochondria-binding dye rhodamine 123 and DNA-binding dye Hoechst 33342). Other procedures for separation that may be used include magnetic separation, using antibody-coated magnetic beads, affinity chromatography, cytotoxic agents joined to a monoclonal antibody or used in conjunction with a monoclonal antibody, including complement and cytotoxins, and “panning” with antibody attached to a solid matrix or any other convenient technique. Techniques providing accurate separation include flow cytometry (e.g., flow cytometry using a plurality of color channels, low angle and obtuse light scattering detecting channels, impedance channels).

A large proportion of differentiated cells may be removed from a sample using a relatively crude separation, where major cell population lineages of the hematopoietic system, such as lymphocytic and myelomonocytic, are removed, as well as lymphocytic populations, such as megakaryocytic, mast cells, eosinophils and basophils. Usually, at least about 70 to 90 percent of the hematopoietic cells will be removed.

The purified stem cells have low side scatter and low to medium forward scatter profiles by FACS analysis. Cytospin preparations show the enriched stem cells to have a size between mature lymphoid cells and mature granulocytes. Cells may be selected based on light-scatter properties as well as their expression of various cell surface antigens.

Preferably, cells are initially separated by a coarse separation, followed by a fine separation, with positive selection of a marker associated with stem cells and negative selection for markers associated with lineage committed cells. Compositions highly enriched in stem cells may be achieved in this manner.

Purified or partially purified stem cells are then administered to the subject. Administration may be local (e.g., by direct administration to tissue of interest) or may be systemic.

### Polynucleotide Therapy

If desired, nucleic acid molecules that encode therapeutic polypeptides are delivered to stem cells, such as bone marrow-derived stem cells, hematopoietic stem cells, their precursors, or progenitors. In other approaches, nucleic acid molecules are delivered to cells of a tissue (e.g., pancreatic tissue, liver tissue, heart tissue, etc.). The nucleic acid molecules must be delivered to the cells of a subject in a form in which they can be taken up so that therapeutically effective levels of the therapeutic polypeptide (e.g., stem cell recruiting factor, such as SDF-1; a hepatocyte growth factor; a cardiocyte growth factor; etc.) or fragment thereof can be produced.

A variety of expression systems exists for the production of therapeutic polypeptides. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof

One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

If desired, a vector expressing stem cell recruiting factors is administered to a tissue or organ. SDF-1 (also called PBSF) (Campbell et al. (1998) Science 279(5349):381-4), 6-C-kine (also called Exodus-2), and MIP-3β (also called ELC or Exodus-3) induced adhesion of most circulating lymphocytes, including most CD4⁺ T cells; and MIP-3α (also called LARC or Exodus-1) triggered adhesion of memory, but not naive, CD4⁺ T cells. Tangemann et al. (1998) J. Immunol. 161:6330-7 disclose the role of secondary lymphoid-tissue chemokine (SLC), a high endothelial venule (HEV)-associated chemokine, with the homing of lymphocytes to secondary lymphoid organs. Campbell et al. (1998) J. Cell Biol 141(4):1053-9 describe the receptor for SLC as CCR7, and that its ligand, SLC, can trigger rapid integrin-dependent arrest of lymphocytes rolling under physiological shear.

In still other approaches, a vector encoding a polypeptide characteristically expressed in a cell of interest is introduced to a stem cell of the invention.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a stem cell recruiting factor, variant, or a fragment thereof, can be cloned into a retroviral vector and expression can be driven from its endogenous promoter, from the retroviral long terminal repeat, or from a promoter specific for a tissue or cell of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer a therapeutic polynucleotide in pancreas, liver, heart, or another tissue or organ of interest.

Non-viral approaches can also be employed for the introduction of a therapeutic to a cell of a subject (e.g., a cell or tissue). For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTR) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified stem cell and/or in a cell of the tissue having a deficiency in cell number. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the subject.

In addition to at least one promoter and at least one heterologous nucleic acid encoding the therapeutic agent, the expression vector preferably includes a selection gene, for example, a neomycin resistance gene, for facilitating selection of stem cells that have been transfected or transduced with the expression vector.

If desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant stem cell recruiting factor, variant, or fragment thereof, either directly to the site of a potential or actual disease-affected tissue or systemically (for example, by any conventional recombinant protein administration technique). The dosage of the administered protein depends on a number of factors, including the size and health of the individual subject. For any particular subject, the specific dosage regimes should be adjusted over time according to the individual need and the professional judgment of the person administering or supervising the administration of the compositions.

### Combination Therapies

Compositions and methods of the invention may be administered alone or in any combination. Preferred combinations include a combination of celastrol and celastrol derivatives in combination with geldanamycin and geldanamycin analogs (e.g., 17-AAG); celastrol and celastrol derivatives in combination with triptolide; and celastrol and celastrol derivatives in combination with a TE-140 peptide having an amino acid sequence described herein. If desired, agents of the invention are administered in combination with any standard therapy known in the art. For example, an agent that activates and/or mobilizes stem cells in bone marrow (e.g., sca1⁺, cd45⁺ and/or cd34⁺ cells) is administered together with an agent that promotes the recruitment, survival, proliferation or transdifferentiation of a stem cell (e.g., a hematopoietic stem cell or other bone marrow derived stem cell or progenitor thereof). Such agents include collagens, fibronectins, laminins, integrins, angiogenic factors, anti-inflammatory factors, glycosaminoglycans, vitrogen, antibodies and fragments thereof, functional equivalents of these agents, and combinations thereof.

Compositions and methods of the invention may be administered in combination with any standard therapy known in the art. For example, celastrol, or structural or functional analogs or derivatives thereof may optionally be administered in combination with conventional therapeutics for the treatment of diabetes (e.g., insulin). If desired, an agent that induces tissue repair or regeneration in an organ of interest (e.g., pancreas, liver, heart, etc.) or prevents or reduces cell death in a tissue or organ of interest (e.g., pancreas, liver, heart, etc.) is administered together with an agent that promotes the recruitment, survival, proliferation or differentiation of a stem cell (e.g., a hematopoietic stem cell or other bone marrow derived stem cell or progenitor thereof).

In other embodiments, an agent of the invention is administered in combination with other agents that enhance bone marrow-derived stem cell mobilization, including cytoxan, cyclophosphamide, VP-16, TE-140 peptide (e.g., 4F-benzoyl-TN14003), and cytokines such as GM-CSF, G-CSF or combinations thereof.

Combinations of the invention may be administered concurrently or within a few hours, days, or weeks of one another. In one approach, a compound of the invention is administered prior to, concurrently with, or following administration of a conventional therapeutic described herein. In some embodiments, it may be desirable to mobilize a bone marrow-derived cell prior to administering an agent of the invention, where such mobilization increases the number of stem cells recruited to the tissue. In other embodiments, it may be preferable to administer the agent that mobilizes a bone marrow-derived cell concurrently with or following (e.g., within 1, 2, 3, 5 or 10 hours) of agent administration.

### Kits

The invention provides kits for tissue repair or for the activation and/or mobilization of bone marrow derived stem cells (e.g., sca1⁺, cd45⁺ and/or cd34⁺ expressing cells). The invention also provides kits for the treatment or prevention of a disease, disorder, or symptoms thereof associated with a deficiency in cell number (e.g., diabetes, liver failure, lung disease, chronic obstructive pulmonary disease, heart disease, or another disease or disorder characterized by excess cell death or a deficiency in cell number). In one embodiment, the kit includes a pharmaceutical pack comprising an effective amount of an agent described herein or combinations described herein. Preferably, the agents are present in unit dosage form. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject in need thereof. The instructions will generally include information about the use of the compounds for the treatment or prevention of a disease or disorder amenable to treatment with a stem cell (e.g., liver failure, chronic obstructive pulmonary disease, heart failure, diabetes). In other embodiments, the instructions include at least one of the following: description of the compound or combination of compounds; dosage schedule and administration for tissue repair, cell death prevention, or for activating sca1⁺, cd45⁺ and cd34⁺ stem cells in the bone marrow, and mobilizing these cells into the peripheral blood; dosage schedule and administration for treatment of a disease described herein, such as diabetes, a pancreatic disorder, acute liver failure, heart disease, myocardial infarction, or any other disease characterized by a deficiency in cell number or an increase in cell death or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

### Example 1

### Triptolide Activated Stem Cell Populations in Blood at 400 μg/kg

Triptolide was administered to C57BL6/J mice to determine the effect of the drug on stem cell populations in blood. Mice received either a placebo or triptolide (400 μg/kg) by intraperitoneal injection. Blood samples were taken from both groups of mice at 24, 48, and 72 hours after administration of the placebo or triptolide. The blood samples were analyzed by FACS for cells expressing stem cell markers. Cells expressing the stem cell markers CD34, CD45, and Sca-1 were quantitated as a percent of the total population to observe the effect of triptolide treatment.

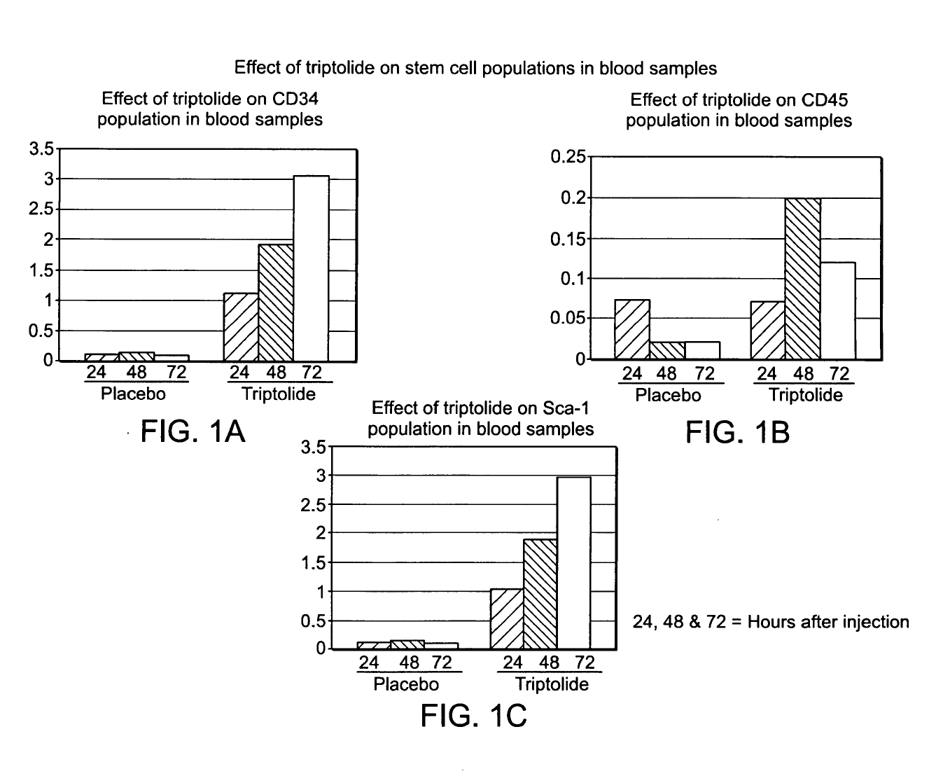

Blood samples showed an increase in the population of cd34⁺, cd45⁺, or sca-1⁺ stem cells compared to samples from untreated mice (FIGS. 1A-1C). At 24 hours post treatment, CD34-expressing cells represented nearly 1% of the population of blood cells (FIG. 1A). This effect was a large increase over the percentage of CD34-expressing cells observed in the blood of control mice. The effect of triptolide resulted in a steady increase in the representation of cd34⁺ cells in the blood up to 72 hours post treatment. Samples taken at 48 hours and 72 hours post treatment, showed a steady increase in the percentage of cd34⁺ cells to nearly 2% and about 3% of cells in blood, respectively.

Triptolide treated mice also showed increases in cd45⁺ stem cells over untreated mice at 48 hours and 72 hours post treatment (FIG. 1B). At 48 hours post treatment, the percentage of cd45⁺ cells increased to about 0.2% of the cells in blood, in contrast to the low percentage of cells found in untreated mice.

At 24 hours post treatment, Sca-1⁺ represented nearly 1% of the population of blood cells (FIG. 1C). This effect was a large increase over the percentage of Sca-1⁺ cells observed in blood of control mice, which were detectable at very low levels. The treatment with triptolide continued to increase the representation of Sca-1 cells in the blood steadily up to 72 hours post treatment. Samples taken at 48 hours and 72 hours post treatment showed a steady increase in the percentage of Sca-1⁺ cells to nearly 2% and nearly 3% of cells in blood, respectively. This analysis shows that triptolide stimulates the increase of stem cell populations in blood.

### Example 2

### Triptolide Activated Stem Cell Populations in Bone Marrow

Triptolide was administered to C57BL6/J mice to determine the effect of the drug on stem cell populations in bone marrow. Mice received either a placebo or triptolide (400 μg/kg) by intraperitoneal injection. Bone marrow samples were taken from both groups of mice at 24, 48, and 72 hours after administration of the placebo or triptolide. The bone marrow samples were analyzed by FACS for cells expressing stem cell markers. Cells expressing the stem cell markers CD34, CD45, and Sca-1, were quantitated as a percent of the total population to observe the effect of triptolide treatment.

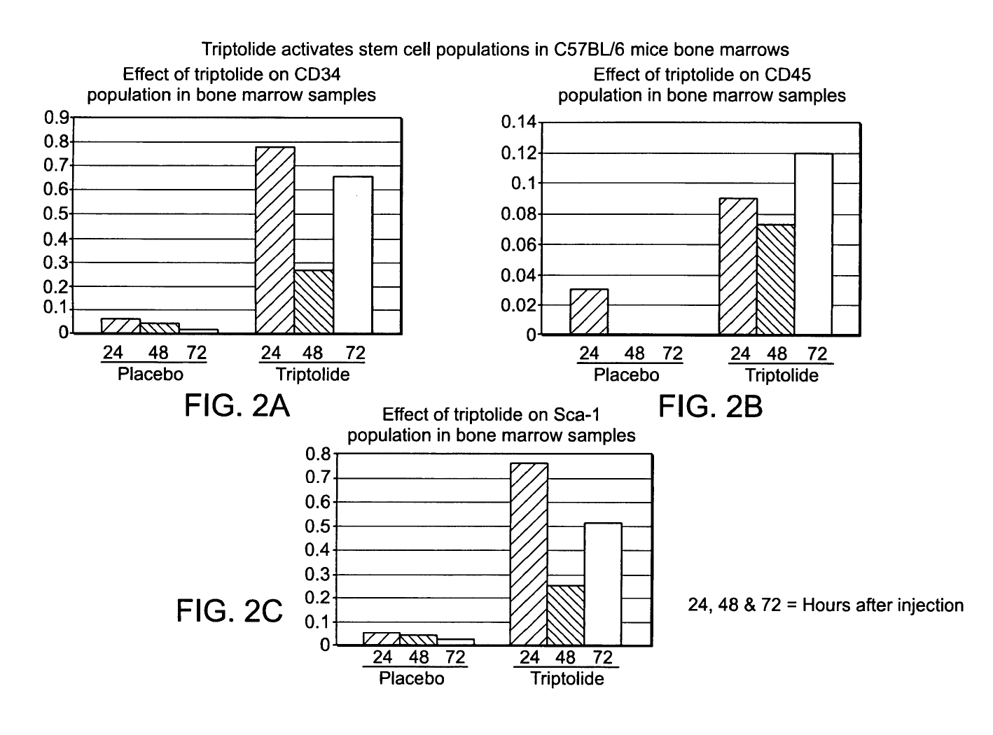

For all stem cell markers evaluated, bone marrow samples showed a noticeable increase in the population of stem cells in triptolide treated mice compared to samples from untreated mice (FIGS. 2A-2C). At 24 hours post treatment, cd34⁺ represented nearly 0.8% of the population of bone marrow cells (FIG. 2A). This effect was nearly a 16-fold increase over the percentage of cd34⁺ cells in bone marrow found in the control. The effect of triptolide was evident over the time course of 72 hours. At 48 hours post treatment 0.3% of cells in triptolide-treated mice were cd34⁺, which represents a 6-fold increase in the percentage of cd34⁺ cells compared to control mice. At 72 hours post treatment the percentage of CD34 cells in bone marrow was 0.65%, which was much greater than the percentage found in control mice.

Similarly, triptolide treated mice showed increases in cd45⁺ stem cells over untreated mice up to 72 hours post treatment (FIG. 2B). At 24 hours post treatment, triptolide increased the percentage of cd45⁺ cells by about 3-fold, when compared to the untreated control mice. At 48 hours and 72 hours post treatment, the percentage of cd45⁺ cells continued to exhibit high levels of representation in bone marrow, about 0.07% and 0.12%, respectively. In contrast, cd45⁺ cells were present at much lower levels in bone marrow samples from untreated mice.

Triptolide treated mice also showed increases in sca-1⁺ stem cells in bone marrow over untreated mice up to 72 hours post treatment (FIG. 2C). At 24 hours post treatment, bone marrow from triptolide treated mice exhibited high representation of sca-1⁺ stem cells in the cell population at 0.75%, compared to about 0.05% in untreated mice, an approximately 15-fold increase in the proportion of Sca-1 stem cells. At 48 hours post treatment sca-1⁺ cells represented 0.25% of the cell population, which was clearly higher than the percentage detected in control mice. At 72 hours post treatment the percentage of Sca-1⁺ cells in bone marrow was still maintained at about 0.5% in triptolide treated mice, while Sca-1⁺ cells were present at much lower levels in the bone marrow of the control mice. This analysis shows that triptolide stimulated an increase in stem cell populations in bone marrow.

### Example 3

### Celastrol and Celastrol Analogs Activated Stem Cell Populations in Bone Marrow

Celastrol was administered to C57BL6/J mice to determine the effect of the drug on stem cell populations in bone marrow. Mice received either a placebo or celastrol (2.5 mg/kg) by intraperitoneal injection. Bone marrow samples were taken from both groups of mice at 24, 48, and 72 hours after administration of the placebo or celastrol. The bone marrow samples were analyzed by FACS for cells expressing stem cell markers. Cells expressing the stem cell markers CD34, CD45, and Sca-1, were quantitated as a percent of the total population to observe the effect of celastrol treatment.

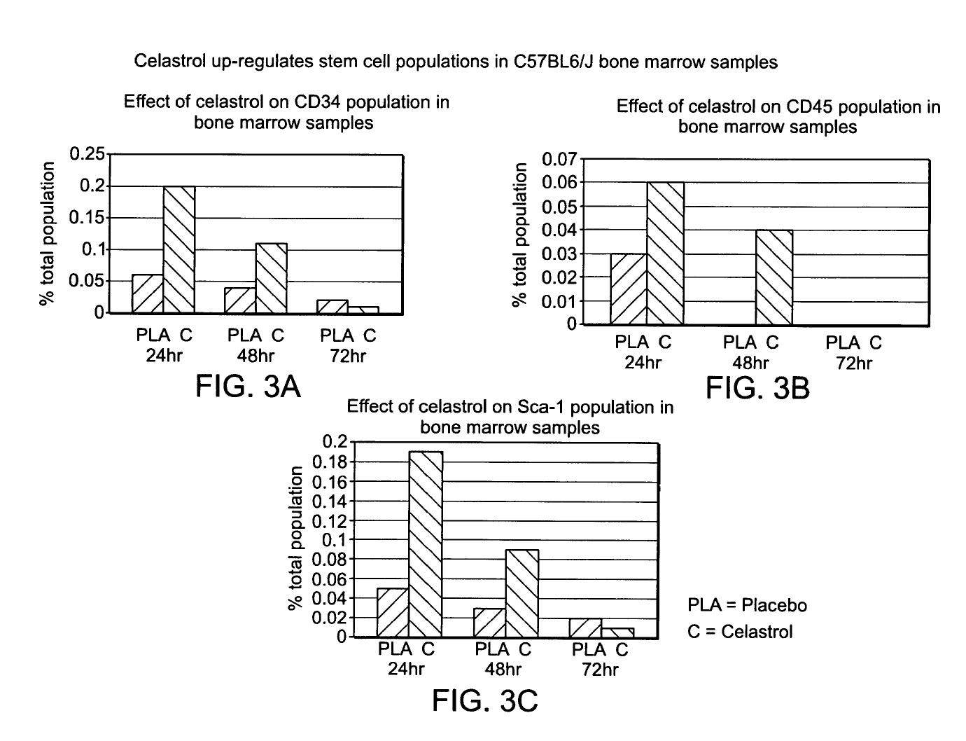

For all stem cell markers evaluated, bone marrow samples showed an increase in the population of cd34⁺, cd45⁺, or Sca-1⁺ stem cells in celastrol treated mice compared to samples from untreated mice (FIGS. 3A-3C). At 24 hours post treatment, it was observed that cd34⁺ cells represented 0.2% of the population of bone marrow cells (FIG. 3A). This effect was about a 4-fold increase over the percentage of cd34⁺ cells in bone marrow found in the control. The effect of celastrol was greatest when observed at 24 hours. By 48 hours there was still about a 2-fold increase in the percentage of cd34⁺ cells in celastrol treated mice compared to the control mice.

Similarly, at 24 hours post treatment, celastrol increased the percentage of cd45⁺ cells by about 2-fold, when compared to the untreated control mice (FIG. 3B). At 48 hours post treatment, CD45 cells were undetectable in mice receiving the placebo. In contrast, an increase in CD45 population was still detectable at 48 hours post treatment in the celastrol treated mice.

Bone marrow from celastrol treated mice also exhibited an approximately 4-fold increase in the proportion of Sca-1⁺ stem cells in bone marrow compared to untreated mice (FIG. 3C), which had decreased to about a 3-fold increase by 48 hours. This analysis shows that celastrol stimulates the increase of stem cell populations in bone marrow. Similar results were observed for the geldanamycin analog 17-AAG. The increase in cd34⁺, cd45⁺, and sca-1⁺ cells observed following celastrol and geldanamycin treatment exceeded that observed using GM-CSF, which is conventionally used for stem cell activation.

Celastrol analogs were administered to C57BL6/J mice to determine the effect of the celastrol analogs on stem cell populations in bone marrow. Celastrol analogs administered included dihydrocelastrol, dihydrocelastrol diacetate, and pristimerol (dihydropristimerin). Mice received either a placebo or celastrol analog (3.0 mg/kg) by intraperitoneal injection. Bone marrow samples were taken from both groups of mice at 24 hours after administration of the placebo or celastrol analog. The bone marrow samples were analyzed by FACS for cells expressing stem cell markers. Cells expressing the stem cell markers CD34, CD45, and Sca-1, were quantitated as a percent of the total population to observe the effect of treatment with celastrol analogs.

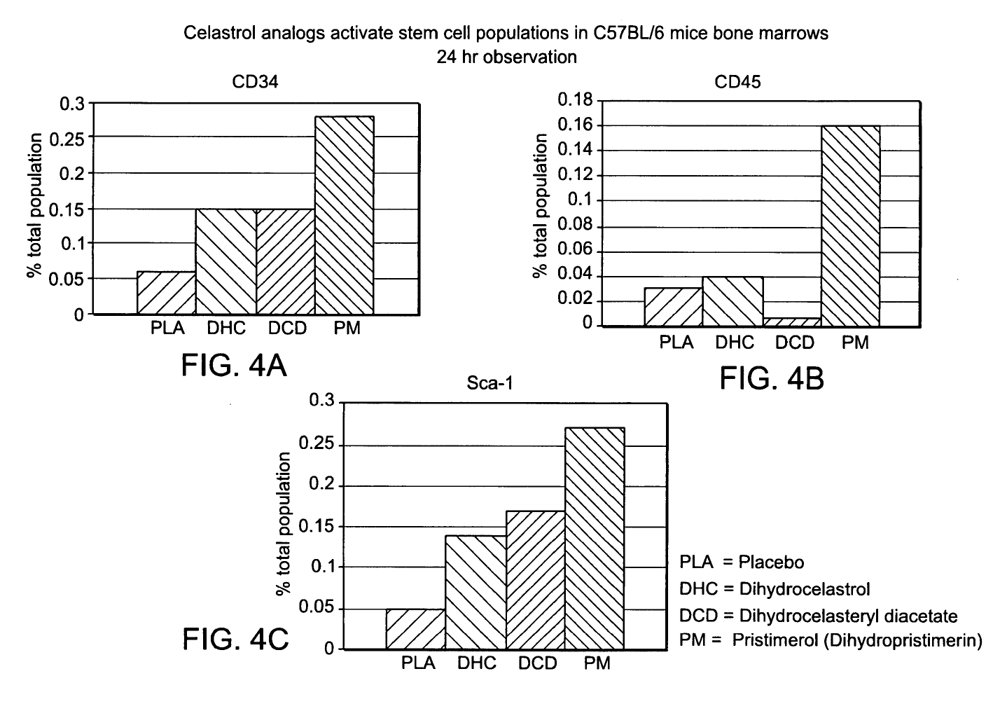

For the stem cell markers evaluated, bone marrow samples showed an increase in the population of cd34⁺ or Sca-1⁺ stem cells in mice treated with celastrol analog compared to samples from untreated mice (FIGS. 4A and 4C). At 24 hours post treatment with dihydrocelastrol or dihydrocelastrol diacetate, it was observed that CD34 cells represented 0.15% of the population of bone marrow cells (FIG. 4A). This effect was about a 3-fold increase over the percentage of cd34⁺ cells in bone marrow found in the control. At 24 hours post treatment with pristimerol, it was observed that cd34⁺ cells represented about 0.28% of the population of bone marrow cells. This effect was a greater than 4-fold increase over the percentage of cd34⁺ cells in bone marrow found in the control.

At 24 hours post treatment, celastrol analogs increased the percentage of cd45⁺ cells when compared to the untreated control mice, except in mice treated with dihydrocelastrol diacetate (FIG. 4B). At 24 hours post treatment with dihydrocelastrol, it was observed that cd45⁺ cells represented 0.04% of the population of bone marrow cells. This effect was a slight increase over the percentage of cd45⁺ cells in bone marrow found in the control. However, at 24 hours post treatment with dihydrocelastrol diacetate, a decrease in the percentage of cd45⁺ cells in bone marrow was observed compared to the untreated control. At 24 hours post treatment with pristimerol, it was observed that cd45⁺ cells represented about 0.16% of the population of bone marrow cells. This effect was a greater than 5-fold increase over the percentage of cd45⁺ cells in bone marrow found in the control.

Bone marrow from mice treated with celastrol analogs also exhibited increases in the proportion of Sca-1⁺ stem cells in bone marrow compared to untreated mice (FIG. 4C). At 24 hours post treatment with dihydrocelastrol, it was observed that Sca-1⁺ cells represented 0.14% of the population of bone marrow cells. This effect was almost a 3-fold increase over the percentage of Sca-1⁺ cells in bone marrow found in the control. At 24 hours post treatment with dihydrocelastrol diacetate, it was observed that Sca-1⁺ cells represented about 0.17% of the population of bone marrow cells. This effect was about a 3-fold increase over the percentage of Sca-1⁺ cells in bone marrow found in the control. At 24 hours post treatment with pristimerol, it was observed that Sca-1⁺ represented about 0.28% of the population of bone marrow cells. This effect was about a 4-5 fold increase over the percentage of Sca-1⁺ cells in bone marrow found in the control. This analysis shows that celastrol analogs stimulate the increase of stem cell populations in bone marrow.

### Example 4

### Celastrol Analogs Activated Stem Cell Populations in Blood

Celastrol analogs were administered to C57BL6/J mice to determine the effect of the celastrol analogs on stem cell populations in blood. Celastrol analogs administered included dihydrocelastrol, dihydrocelastrol diacetate, and pristimerol (dihydropristimerin). Mice received either a placebo or a celastrol analog (3.0 mg/kg) by intraperitoneal injection. Blood samples were taken from both groups of mice at 48 hours after administration of the placebo or a celastrol analog. The blood samples were analyzed by FACS for cells expressing stem cell markers. Cells expressing the stem cell markers CD34, CD45, and Sca-1 were quantitated as a percent of the total population to observe the effect of treatment with a celastrol analog.

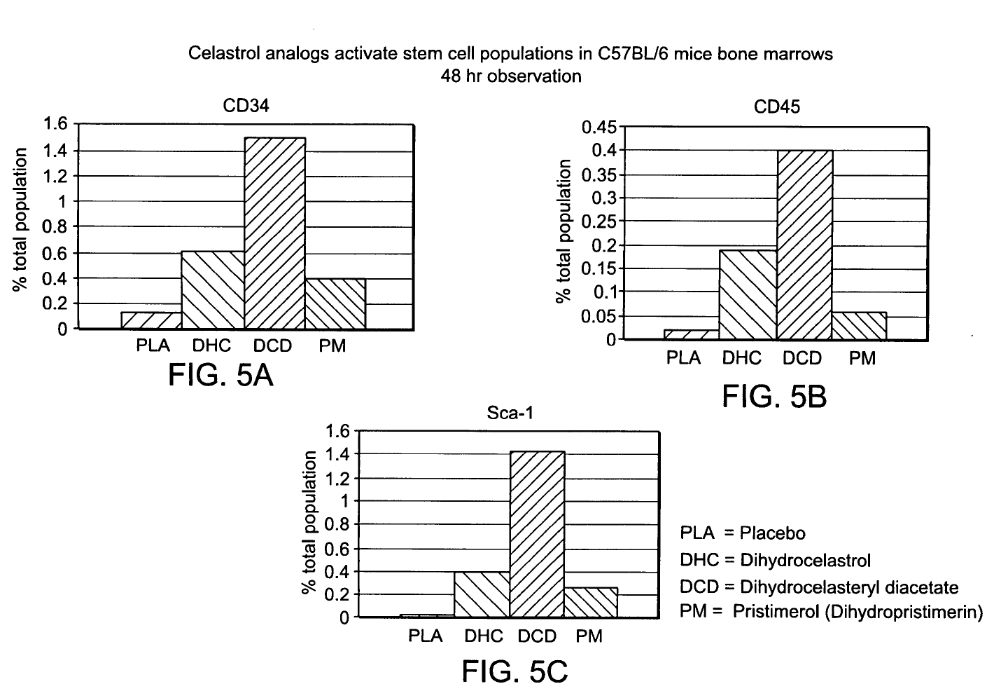

Blood samples showed an increase in the population of cd34⁺ or Sca-1⁺ stem cells in mice treated with celastrol analog compared to samples from untreated mice (FIGS. 5A-5C). At 24 hours post treatment with dihydrocelastrol, it was observed that cd34⁺ represented approximately 0.6% of the population of blood cells (FIG. 5A). This effect was about a 4-fold increase over the percentage of cd34⁺ cells in blood found in the control. At 24 hours post treatment with dihydrocelastrol diacetate, it was observed that cd34⁺ cells represented 1.5% of the population of blood cells. This effect was about a 10-fold increase over the percentage of cd34⁺ cells in blood found in the control. At 24 hours post treatment with pristimerol, it was observed that cd34⁺ cells represented about 0.4% of the population of blood cells (FIG. 5A). This effect was almost a 3-fold increase over the percentage of cd34⁺ cells in blood found in the control.

At 24 hours post treatment, celastrol analogs increased the percentage of cd45⁺ cells in blood when compared to the untreated control mice (FIG. 5B). At 24 hours post treatment with dihydrocelastrol, it was observed that cd45⁺ cells represented about 0.19% of the population of blood cells. This effect was almost a 10-fold increase over the percentage of cd45⁺ cells in blood found in the control. At 24 hours post treatment with dihydrocelastrol diacetate, it was observed that cd45⁺ cells represented about 0.4% of the population of blood cells. This effect was about a 20-fold increase over the percentage of cd45⁺ cells in blood found in the control. At 24 hours post treatment with pristimerol, it was observed that cd45⁺ cells represented about 0.06% of the population of blood cells. This effect was about a 3-fold increase over the percentage of cd45⁺ cells in blood found in the control.

Blood from mice treated with celastrol analogs also exhibited increases in the proportion of Sca-1⁺ stem cells in blood compared to untreated mice (FIG. 5C). At 24 hours post treatment with dihydrocelastrol, it was observed that Sca-1⁺ cells represented 0.4% of the population of blood cells. This effect was about a 10-fold increase over the percentage of Sca-1⁺ cells in blood found in the control. At 24 hours post treatment with dihydrocelastrol diacetate, it was observed that Sca-1⁺ cells represented about 1.4% of the population of blood cells. This effect was about a 36-fold increase over the percentage of Sca-1⁺ cells in blood found in the control. At 24 hours post treatment with pristimerol, it was observed that Sca-1⁺ represented about 0.25% of the population of blood cells. This effect was about a 6-fold increase over the percentage of Sca-1⁺ cells in blood found in the control. This analysis shows that celastrol analogs stimulate the increase of stem cell populations in blood.

### Example 5

### Celastrol and Triptolide Synergistically Activated Stem Cell Populations in Bone Marrow

Celastrol and triptolide was administered to C57BL6/J mice to determine the effect of the drugs on stem cell populations in bone marrow. Mice received either a placebo, celastrol (3.0 mg/kg), triptolide (400 μg/kg), or both celastrol (2.5 mg/kg) and triptolide (200 μg/kg) by intraperitoneal injection. For the mice receiving celastrol and triptolide in combination, the doses of celastrol and triptolide were reduced from those used for either compound alone to reduce any toxic effects from using both compounds in combination. In other experiments treatment of mice with celastrol alone at 3.0 mg/kg or 2.5 mg/kg showed similar effects on stem cell activation. Treatment of mice with triptolide alone at 400 μg/kg or 200 μg/kg also showed similar effects on stem cell activation in other experiments. Bone marrow samples were taken from each group of mice at 24 hours and 48 hours after administration. The bone marrow samples were analyzed by FACS for cells expressing stem cell markers. Cells expressing the stem cell markers CD34, CD45, and Sca-1, were quantitated as a percent of the total population to observe the effect of celastrol treatment.

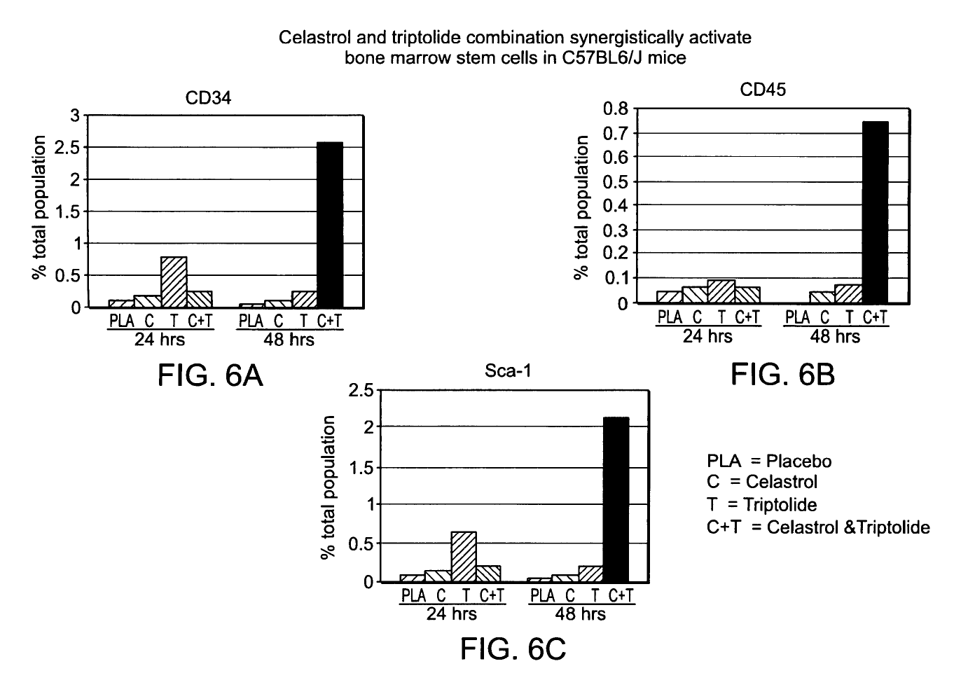

For all stem cell markers evaluated, bone marrow samples showed a synergistic increase in the population of cd34⁺, cd45⁺, or Sca-1⁺ stem cells in mice treated with celastrol and triptolide compared to samples from mice treated with placebo, celastrol alone, or triptolide alone at 48 hours post treatment (FIGS. 6A-6C). At 48 hours post treatment with celastrol and triptolide, it was observed that cd34⁺ cells represented about 2.6% of the population of bone marrow cells (FIG. 6A). In mice treated with placebo, celastrol alone, or triptolide alone cd34⁺ cells represented 0.3% or less of the total bone marrow population at 48 hours post treatment. This effect was a much greater increase in the percentage of cd34⁺ cells in bone marrow over the untreated mice than would have been expected merely from the effects observed in the mice treated with celastrol or triptolide alone at 48 hours post treatment. This effect was also greater than the increases in cd34⁺ cells from either celastrol or triptolide treatment, observed at 24 hours post treatment. The effect of treatment with celastrol and triptolide in combination on cd34⁺ cell population showed about a 10-fold increase at 48 hours post treatment compared to 24 hours post treatment.

Similarly, at 48 hours post treatment, celastrol and triptolide in combination increased the percentage of cd45⁺ cells, when compared to the untreated control mice (FIG. 6B). At 48 hours post treatment with celastrol and triptolide, it was observed that cd45⁺ cells represented more than 0.7% of the population of bone marrow cells. In contrast, mice treated with placebo, celastrol alone, or triptolide alone all showed cd45⁺ cells represented 0.1% or less of the total bone marrow population at either 48 hours or 24 hours post treatment. This effect was a much greater increase in the percentage of cd45⁺ cells in bone marrow over the untreated mice than would have been expected merely from the effects observed in the mice treated with celastrol or triptolide alone at either 48 hours or 24 hours post treatment. The effect of treatment with celastrol and triptolide in combination on cd45⁺ cell population showed a greater than 7-fold increase at 48 hours post treatment compared to 24 hours post treatment.

Bone marrow from mice treated with celastrol and triptolide in combination also exhibited a significant increase in the proportion of Sca-1⁺ stem cells in bone marrow compared to untreated mice at 48 hours post treatment (FIG. 6C). At 48 hours post treatment with celastrol and triptolide, it was observed that Sca-1⁺ cells represented at least 2.5% of the population of bone marrow cells. This effect was a much greater increase in the percentage of Sca-1⁺ cells in bone marrow over the untreated mice than would have been expected merely from the effects observed in the mice treated with celastrol or triptolide alone at 48 hours post treatment. This effect was also greater than the increases in cd34⁺ cells from either celastrol or triptolide treatment, observed at 24 hours post treatment. The effect of treatment with celastrol and triptolide in combination on Sca-1⁺ cell population showed a greater than 10-fold increase at 48 hours post treatment compared to 24 hours post treatment. This analysis shows that celastrol in combination with triptolide synergistically stimulates the increase of stem cell populations in bone marrow.

### Example 6

### Celastrol and TE-140 Peptide Synergistically Activated Stem Cell Populations in Bone Marrow

Celastrol and TE-140 peptide (H-Arg-Arg-Nal-Cys-Tyr-Arg-Lys-dLys-Pro-Tyr-Arg-Cit-Cys-Arg-OH) were administered to C57BL6/J mice to determine the effect of the drugs on stem cell populations in bone marrow. Mice received either a placebo, celastrol (3.0 mg/kg), TE-140 peptide (250 μg/kg), or both celastrol (3.0 mg/kg) and TE-140 peptide (250 μg/kg), by intraperitoneal injection. Bone marrow samples were taken from all groups of mice at 24 hours after administration. The bone marrow samples were analyzed by FACS for cells expressing stem cell markers. Cells expressing the stem cell markers CD34, CD45, and Sca-1, were quantitated as a percent of the total population to observe the effect of celastrol treatment.

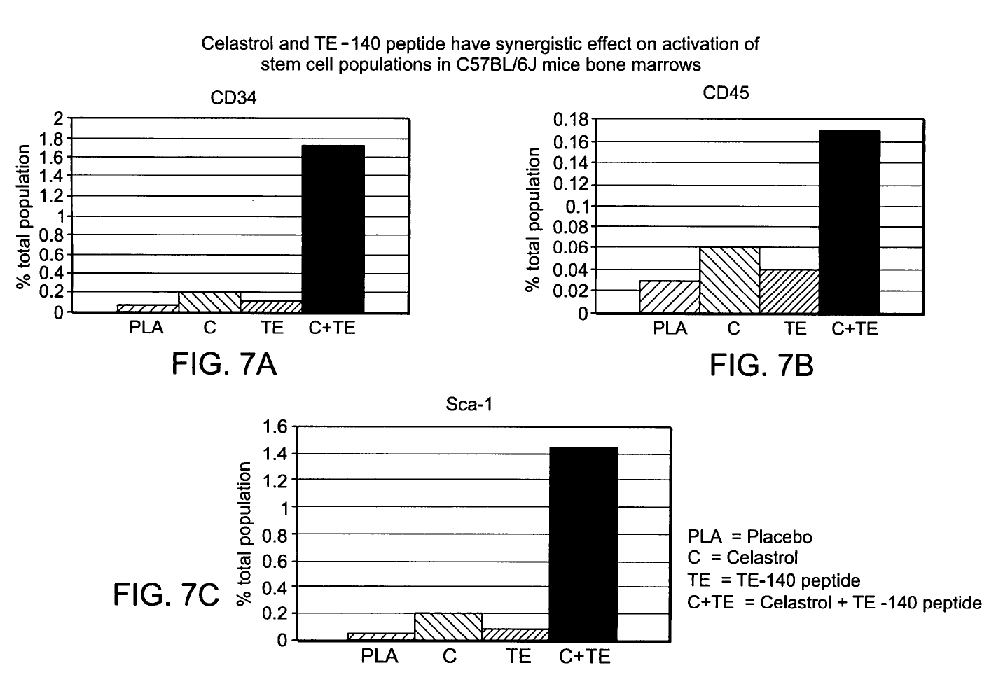

For all stem cell markers evaluated, bone marrow samples showed a synergistic increase in the population of cd34⁺, cd45⁺, or Sca-1⁺ stem cells in mice treated with celastrol and TE-140 peptide compared to samples from mice treated with placebo, celastrol alone, or TE-140 peptide alone (FIGS. 7A-7C). At 24 hours post treatment with celastrol and TE-140 peptide, it was observed that cd34⁺ cells represented about 1.7% of the population of bone marrow cells (FIG. 7A). In contrast, mice treated with placebo, celastrol alone, or TE-140 peptide alone all showed that cd34⁺ cells represented 0.2% or less of the total bone marrow population. This effect was a much greater increase in the percentage of cd34⁺ cells in bone marrow over the untreated mice than would have been expected merely from the effects observed in the mice treated with celastrol or TE-140 peptide alone.

Similarly, at 24 hours post treatment, celastrol and TE-140 peptide in combination increased the percentage of cd45⁺ cells greater than 5-fold, when compared to the untreated control mice (FIG. 7B). This effect was a much greater increase in the percentage of cd45⁺ cells in bone marrow over the untreated mice than would have been expected merely from the effects observed in the mice treated with celastrol or TE-140 peptide alone.

Bone marrow from mice treated with celastrol and TE-140 peptide in combination also exhibited a significant increase in the proportion of Sca-1⁺ stem cells in bone marrow compared to untreated mice (FIG. 7C). At 24 hours post treatment with celastrol and TE-140 peptide, it was observed that Sca-1⁺ cells represented at least 1.4% of the population of bone marrow cells. In contrast, mice treated with placebo, celastrol alone, or TE-140 peptide alone all showed Sca-1⁺ cells represented 0.2% or less of the total bone marrow population. This effect was a much greater increase in the percentage of Sca-1⁺ cells in bone marrow over the untreated mice than would have been expected merely from the effects observed in the mice treated with celastrol or TE-140 peptide alone. This analysis shows that celastrol in combination with TE-140 peptide synergistically stimulates the increase of stem cell populations in bone marrow.

Sequences of TE-140 peptides useful in the methods of the invention are provided in the Table below.

TE-140 Peptide Sequences
TE-140
H-Arg-Arg-Nal-Cys-Tyr-Arg-Lys-DLys-Pro-Tyr-Arg-
Cit-Cys-Arg-OH
TE14001
H-Glu-Arg-Nal-Cys-Tyr-Arg-Lys-DLys-Pro-Tyr-Arg-
Cit-Cys-Arg-OH
TE14002
H-Arg-Glu-Nal-Cys-Tyr-Arg-Lys-DLys-Pro-Tyr-Arg-
Cit-Cys-Arg-OH
TE14003
H-Arg-Arg-Nal-Cys-Tyr-Glu-Lys-DLys-Pro-Tyr-Arg-
Cit-Cys-Arg-OH
TE14004
H-Arg-Arg-Nal-Cys-Tyr-Arg-Glu-DLys-Pro-Tyr-Arg-
Cit-Cys-Arg-OH
TE14005
H-Arg-Arg-Nal-Cys-Tyr-Arg-Lys-DGlu-Pro-Tyr-Arg-
Cit-Cys-Arg-OH
TE14006
H-Arg-Arg-Nal-Cys-Tyr-Arg-Lys-DLys-Pro-Tyr-Glu-
Cit-Cys-Arg-OH
TE14007
H-Arg-Arg-Nal-Cys-Tyr-Arg-Lys-DLys-Pro-Tyr-Arg-
Cit-Cys-Glu-OH
TE14011
H-Arg-Arg-Nal-Cys-Tyr-Cit-Lys-DGlu-Pro-Tyr-Arg-
Cit-Cys-Arg-NH₂
TE14012
H-Arg-Arg-Nal-Cys-Tyr-Glu-Lys-DCit-Pro-Tyr-Arg-
Cit-Cys-Arg-NH₂
TE14013
H-Arg-Arg-Nal-Cys-Tyr-Glu-Lys-DGlu-Pro-Tyr-Arg-
Cit-Cys-Arg-NH₂
TE14014
H-Glu-Arg-Nal-Cys-Tyr-Cit-Lys-DGlu-Pro-Tyr-Arg-
Cit-Cys-Arg-NH₂
TE14015
H-Arg-Arg-Nal-Cys-Tyr-Cit-Lys-DGlu-Pro-Glu-Arg-
Cit-Cys-Arg-NH₂
TE14016
H-Arg-Arg-Nal-Cys-Tyr-Cit-Lys-DGlu-Pro-Tyr-Arg-
Glu-Cys-Arg-NH₂
TC14003
H-Arg-Arg-Nal-Cys-Tyr-Cit-Lys-DLys-Pro-Tyr-Arg-
Cit-Cys-Arg-OH
TC14005
H-Arg-Arg-Nal-Cys-Tyr-Arg-Lys-DCit-Pro-Tyr-Arg-
Cit-Cys-Arg-OH
TN14003
H-Arg-Arg-Nal-Cys-Tyr-Cit-Lys-DLys-Pro-Tyr-Arg-
Cit-Cys-Arg-NH₂
TN14005
H-Arg-Arg-Nal-Cys-Tyr-Arg-Lys-DCit-Pro-Tyr-Arg-
Cit-Cys-Arg-NH₂
TC14012
H-Arg-Arg-Nal-Cys-Tyr-Cit-Lys-DCit-Pro-Tyr-Arg-
Cit-Cys-Arg-NH₂
TC14013
H-Arg-Arg-Nal-Cys-Tyr-Cit-Lys-DLys-Pro-Tyr-Cit-
Cit-Cys-Arg-OH
TC14014
H-Arg-Arg-Nal-Cys-Tyr-Cit-Lys-DLys-Pro-Tyr-Cit-
Cit-Cys-Arg-NH₂
TC14015
H-Cit-Arg-Nal-Cys-Tyr-Cit-Lys-DLys-Pro-Tyr-Arg-
Cit-Cys-Arg-OH
TC14016
H-Cit-Arg-Nal-Cys-Tyr-Cit-Lys-DLys-Pro-Tyr-Arg-
Cit-Cys-Arg-NH₂
TC14017
H-Cit-Arg-Nal-Cys-Tyr-Arg-Lys-DCit-Pro-Tyr-Arg-
Cit-Cys-Arg-OH
TC14018
H-Cit-Arg-Nal-Cys-Tyr-Arg-Lys-DCit-Pro-Tyr-Arg-
Cit-Cys-Arg-NH₂
TC14019
H-Arg-Arg-Nal-Cys-Tyr-Arg-Lys-DCit-Pro-Tyr-Cit-
Cit-Cys-Arg-OH
TC14020
H-Arg-Arg-Nal-Cys-Tyr-Arg-Lys-DCit-Pro-Tyr-Cit-
Cit-Cys-Arg-NH₂
TC14021
H-Cit-Arg-Nal-Cys-Tyr-Arg-Lys-DLys-Pro-Tyr-Cit-
Cit-Cys-Arg-OH
TC14022
H-Cit-Arg-Nal-Cys-Tyr-Arg-Lys-DLys-Pro-Tyr-Cit-
Cit-Cys-Arg-NH₂

### Example 7

### Celastrol and 17-AAG Modulated Production of Cytokines in Peripheral Blood-Like Mesenchymal Stem Cells

Celastrol or 17-allylamino-17-demethoxygeldanamycin (17-AAG) were administered to C57BL6/J mice to determine the effect of the drug on cytokine production in peripheral blood-like mesenchymal stem cells. Mice received either a placebo, celastrol (3.0 mg/kg), or 17-AAG (10 mg/kg) by intraperitoneal injection. Blood samples were taken from all groups of mice at 0, 1, 3, 6, 24, 48, and 72 hours after administration of the placebo, celastrol, or 17-AAG. The blood samples were analyzed for cytokine production, including the production of granulocyte-macrophage colony-stimulating factor (GM-CSF), interferon-gamma (IFN-γ), interleukin-10 (IL-10), interleukin-12 subunit beta (IL-12p40), interleukin-12 heterodimer (IL-12p70), interleukin-12 homodimer (IL-12p80), vascular endothelial growth factor (VEGF), tumor necrosis factor-alpha (TNF-α), keratinocyte derived chemokine (KC), RANTES (Regulated upon Activation, Normal T-cell Expressed, and Secreted; CCL5), monocyte chemotactic protein-1 (MCP 1), macrophage inflammatory protein (MIP1β; CCL4), interleukin-4 (IL-4), interleukin-3 (IL-3), interleukin-2 (IL-2), interleukin-1 beta (IL-1β), interleukin-1 alpha (IL-1α), interleukin-9 (IL-9), interleukin-13 (IL-13), interleukin-17 (IL-17), interleukin-6 (IL-6), and interleukin-5 (IL-5). Blood serum was analyzed using a commercially available kit (e.g., Mouse cytokine/chemokine panel: 21-Plex; Millipore). Blood serum (50 μl) was assayed in duplicate using a commercially available cytokine/chemokine detection kit from Millipore following the protocol given with the product information. The final measurement was performed using a commercially available detector (e.g., computer-operated Luminex 100 IS system) and the results were expressed as pg/ml blood serum sample. Levels of cytokines in blood were quantitated as a concentration of the total volume of the sample.

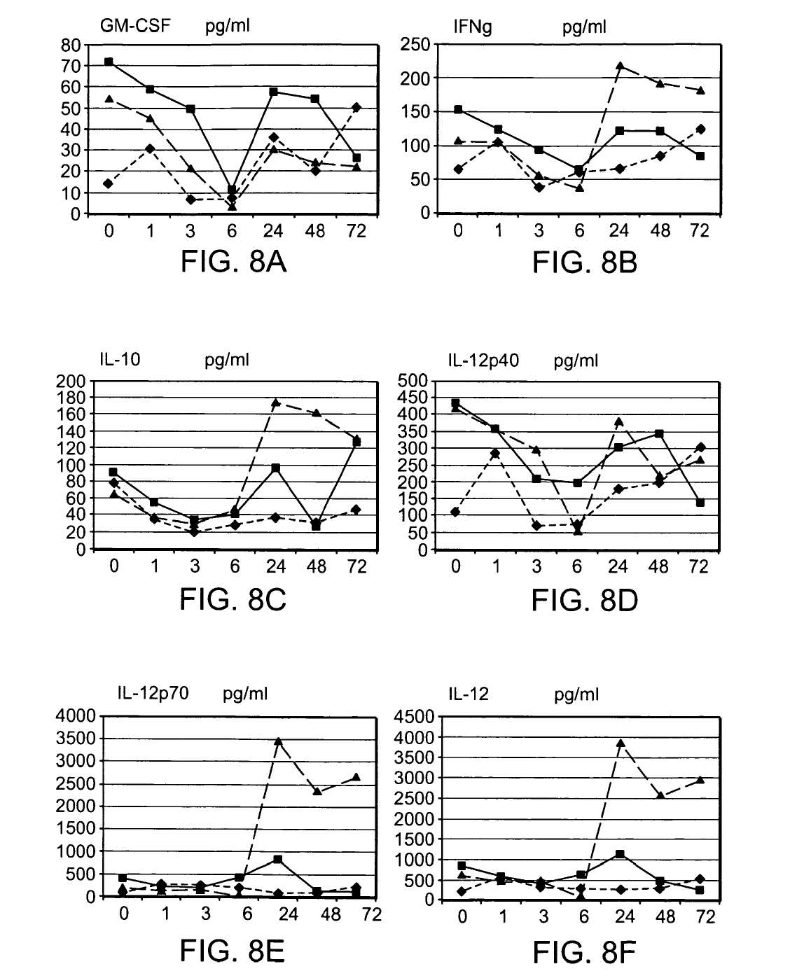

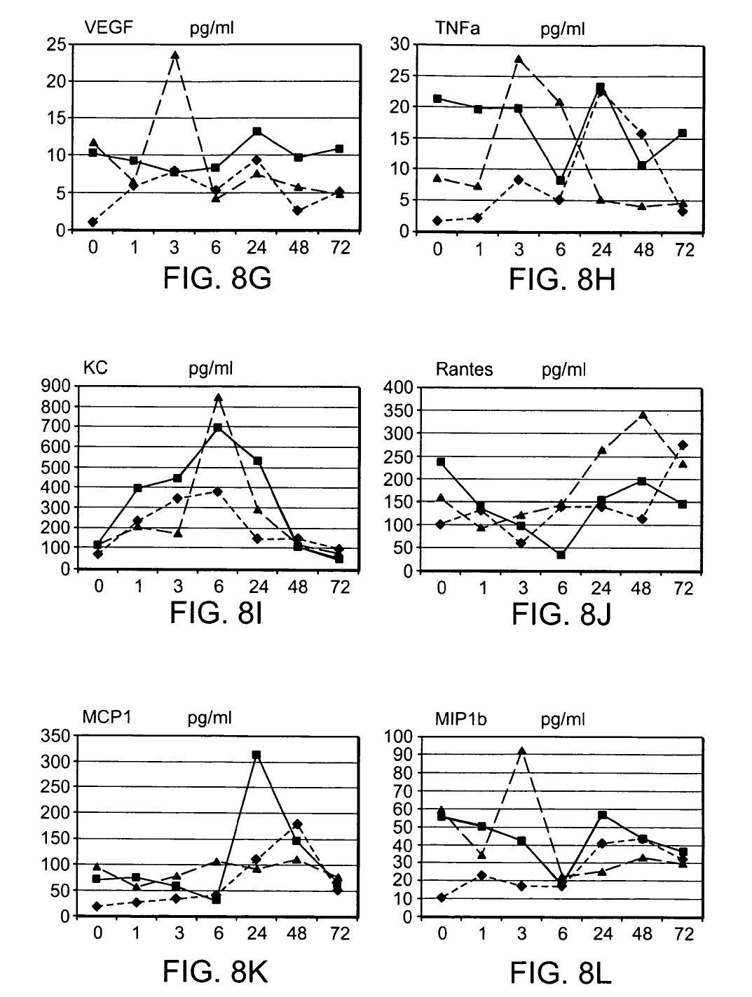

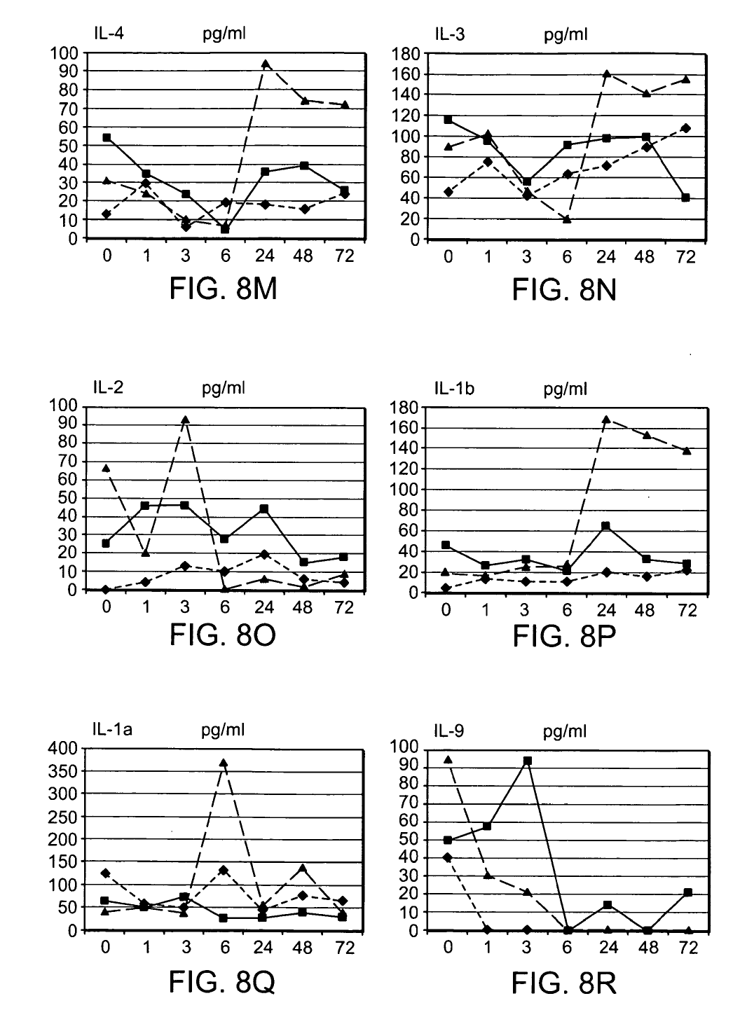

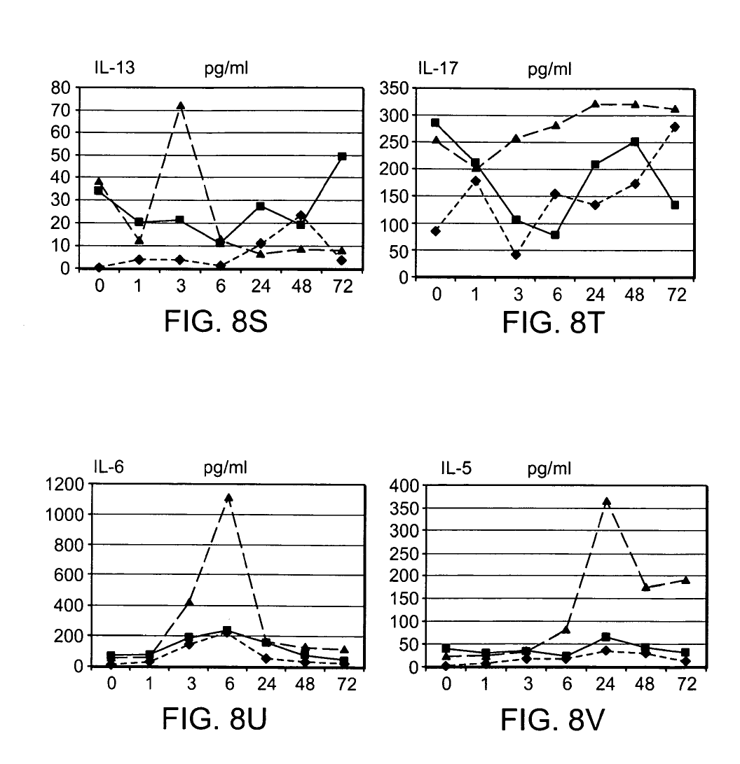

Treatment with 17-AAG resulted in changes in cytokine levels in blood. As observed in the 72 hour time course, the levels of VEGF, TNF-α, MIP1β, IL-2, and IL-13, peaked around 3 hours post treatment with 17-AAG compared to the levels of the respective cytokines in the placebo control (FIGS. 8G, 8H, 8L, 8O, and 8S). By 6 hours post treatement, VEGF, MIP1β, IL-2, and IL-13, had returned to levels seen in the placebo control (FIGS. 8G, 8L, 8O, and 8S). By 24 hours post treatment, TNF-α, the level of TNF-α declined below levels seen in the placebo control, which increased 24 hours post treatment (FIG. 8H). The levels of KC, IL-1α, IL-6 peaked around 6 hours post treatment with 17-AAG, compared to the levels of the respective cytokines in the placebo control, and returned to levels seen in the placebo control by 24 hours post treatment (FIGS. 8I, 8Q, and 8U). The levels of IFN-γ, IL-10, IL-12p70, IL-12p80, IL-4, IL-3, IL-1β, and IL-5 peaked around 24 hours post treatment with 17-AAG and remained elevated up to 72 hours post treatment, compared to the levels of the respective cytokines in the placebo control (FIGS. 8B, 8C, 8E, 8F, 8M, 8N, 8P, and 8V). The level of RANTES peaked around 48 hours post treatment with 17-AAG, compared to the levels of the respective cytokines in the placebo control, and returned to levels seen in the placebo control by 72 hours post treatment (FIG. 8J). The levels of GM-CSF, IL-12p40, and IL-9 were elevated pre treatment with 17-AAG but declined to placebo control levels by 6 hours (FIGS. 8A, 8D, and 8R). The level of IL-12p40 became elevated again at 24 hours compared to placebo control but returned to levels seen in the placebo control by 72 hours post treatment (FIG. 8D). The level of IL-17 was consistently high in mice treated with 17-AAG up to 48 hours post treatment, compared to control mice receiving placebo, but IL-17 in the control levels had risen to that observed in the mice treated with 17-AAG by 72 hours post treatment (FIG. 8T). The level of MCP1 in mice treated with 17-AAG were not significantly different from those in the control mice receiving placebo over the course of the experiment (FIG. 8K). This analysis shows that 17-AAG modulates cytokine production in peripheral blood-like mesenchymal stem cells.

Treatment with celastrol resulted in changes in cytokine levels in blood. As observed in the 72 hour time course, the level of GM-CSF was elevated pre treatment with celastrol but declined to placebo control levels by 6 hours post treatment (FIG. 8A). However the level of GM-CSF became elevated again between 24 and 48 hours post treatment with celastrol, compared to placebo control. The levels of TNF-α and MIP1β were elevated pre treatment with celastrol but declined to placebo control levels by 6 hours (FIGS. 8H and 8L). The level of IL-4 was slightly elevated pre treatment with celastrol but declined to placebo control levels by 1 hour post treatment (FIG. 8M). However the level of GM-CSF became elevated again between 24 and 48 hours post treatment with celastrol, compared to placebo control. The level of IL-3 was slightly elevated pre treatment with celastrol but declined to placebo control levels by 1 hours post treatment (FIG. 8N). However the level of GM-CSF became elevated again 6 to 48 hours post treatment with celastrol, compared to placebo control. The level of IL-9 peaked around 3 hours post treatment with celastrol and remained slightly elevated up to 72 hours post treatment, compared to the levels of the respective cytokines in the placebo control (FIG. 8R). The level of KC peaked around 6 hours post treatment with celastrol, compared to the levels of the respective cytokines in the placebo control, and returned to levels seen in the placebo control by 48 hours post treatment (FIG. 8I). The level of MCP1 peaked around 24 hours post treatment with 17-AAG, compared to the levels of the respective cytokines in the placebo control, and returned to levels seen in the placebo control by 48 hours post treatment (FIG. 8K). The level of IL-10 peaked slightly around 24 hours post treatment with 17-AAG, compared to the levels of the respective cytokines in the placebo control, and had risen again by 72 hours post treatment, compared to control mice receiving placebo (FIG. 8C). The levels of IL-12p70, IL-12p80, and IL-1β also appeared to peak slightly around 24 hours post treatment with 17-AAG, compared to the levels of the respective cytokines in the placebo control, and returned to levels seen in the placebo control by 48 hours post treatment (FIGS. 8E, 8F, and 8P). The level of IL-13 was slightly elevated in mice treated with celastrol up to 48 hours post treatment but had risen again by 72 hours post treatment, compared to control mice receiving placebo (FIG. 8S). The levels of IL-12p40, IL-2, and IL-17 were slightly elevated in mice treated with celastrol throughout the 72 hour time course, compared to control mice receiving placebo (FIGS. 8D, 8O, and 8T). The levels of IFN-γ and VEGF were slightly elevated in mice treated with celastrol throughout the 72 hour time course, compared to control mice receiving placebo (FIGS. 8B and 8G). The level of IL-1α was slightly decreased in mice treated with celastrol throughout the experiment, compared to control mice receiving placebo (FIG. 8Q). The levels of RANTES, IL-6, and IL-5 in mice treated with celastrol were not significantly different from those in the control mice receiving placebo over the course of the experiment (FIGS. 8J, 8U, and 8V). This analysis shows that celastrol modulates cytokine production in peripheral blood-like mesenchymal stem cells.

### Example 8

### Celastrol Restored Normoglycemia in a Mouse Model of Type I Diabetes

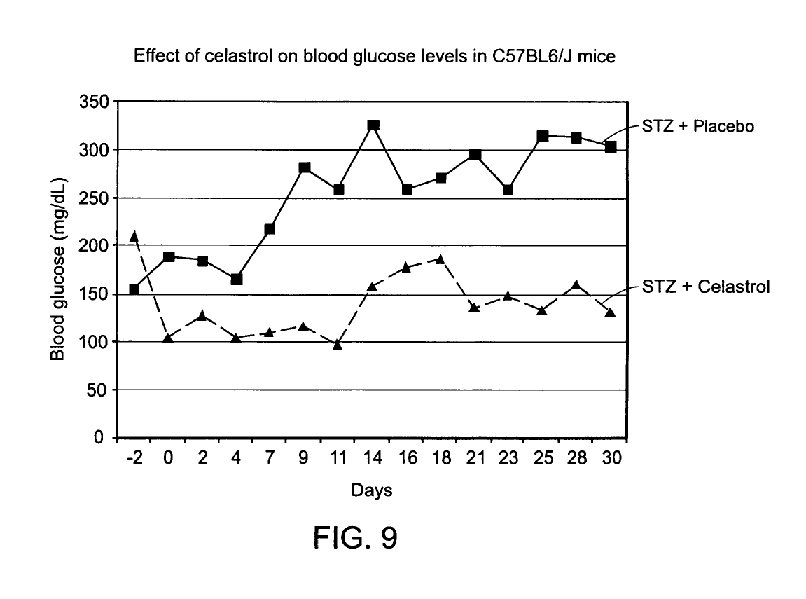

The administration of streptozotocin to induce pancreatic cell death is a well-known model of diabetes, see for example, Like and Rossini, “Streptozotocin-induced pancreatic insulitis: new model of diabetes mellitus.” Science. Jul. 30, 1976; 193(4251):415-7. Fasting C57BL6/J mice received 50 mg/kg streptozotocin (STZ) by intraperitoneal injection at day 0. Two days prior to (−2 day) and concurrently with STZ injection half of the mice also received celastrol by injection (2.5 mg/kg). Celastrol injections continued three times per week throughout the experiment. Blood glucose levels were monitored for 30 days after treatment (FIG. 9). Hyperglycemia was observed in STZ treated mice that received placebo. Blood glucose levels were substantially lower in STZ mice treated with celastrol relative to blood glucose levels in STZ mice injected with a saline placebo. In mice, normoglycemia is typically less than 200 mg/dL blood glucose. Celastrol treatment prior to or post adminstration of streptozotocin gave similar results as celastrol treatment concurrent with administration of streptozotocin.

These results indicate that celastrol rescued pancreatic tissue in mice with STZ induced diabetes. In fact, STZ mice treated with celastrol showed normoglycemia. In contrast, STZ mice that received placebo exhibited marked hyperglycemia.

### Example 9

### Celastrol Rescued Pancreatic Tissue in mice with STZ Induced Diabetes

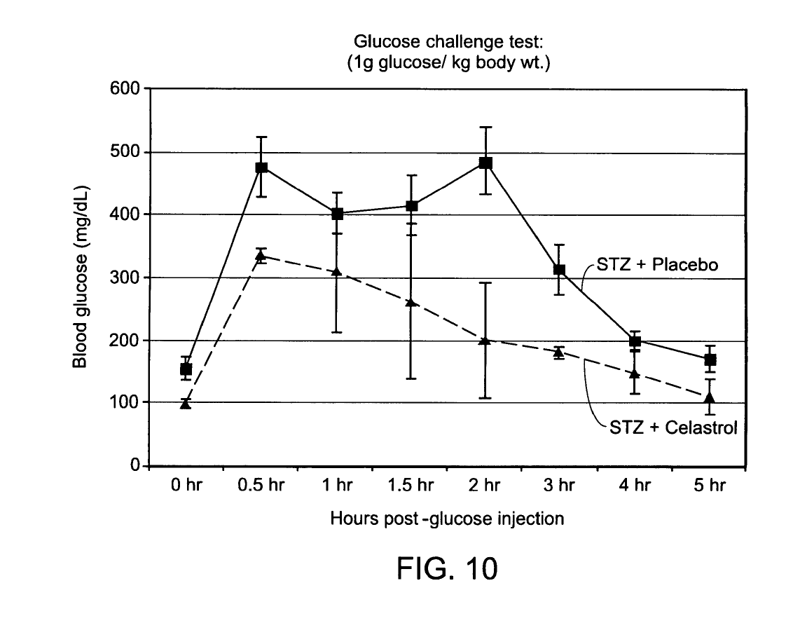

STZ was administered to C57BL6/J mice as described above to induce diabetes. Groups of C57BL6/J mice that received either STZ and placebo or STZ and celastrol were then subjected to a glucose challenge test to assay glucose metabolism. STZ mice received 1 g glucose/kg. Blood glucose levels were monitored for up to 5 hours following administration of glucose. STZ mice treated with placebo exhibited dramatic hyperglycemia in response to glucose challenge. STZ mice treated with placebo showed blood glucose levels at or above 400 mg/dl between 0.5 and 2 hours after glucose challenge. Blood glucose levels never reached these levels in STZ mice treated with celastrol. In fact, mice treated with celastrol were better able to metabolize glucose than STZ mice that received placebo (FIG. 10). Celastrol treatment prior to or post adminstration of streptozotocin gave similar results as celastrol treatment concurrent with administration of streptozotocin. This analysis shows that celastrol rescued pancreatic tissue in mice with STZ induced diabetes.

### Example 10

### Celastrol Rescued Liver Tissue in an In Vivo Model of Acute Liver Failure

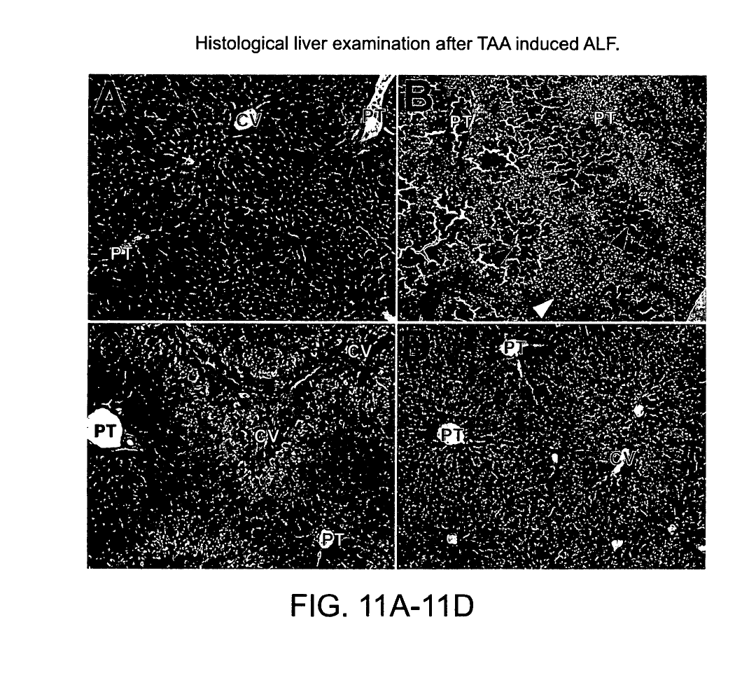

The administration of thiomacetamide (TAA) to induce liver damage is a well-known model of acute liver failure (ALF), see for example, Müller et al., “Thioacetamide-induced cirrhosis-like liver lesions in rats—usefulness and reliability of this animal model.” Exp Pathol. 1988; 34(4):229-36. Groups of C57BL6/J mice received either TAA (1000 mg/kg), TAA (500 mg/kg), or placebo by intraperitoneal injection. The group receiving TAA (500 mg/kg) was divided into two subgroups, and one group received NS by injection and the other group received celastrol by injection (2.5 mg/kg). Mice were sacrificed either one or three days after treatment. The livers of the mice were removed for histological examination (FIGS. 11A-11D).

Liver tissue from mice receiving saline appeared normal under histological examination (FIG. 11A). In contrast, liver parenchyma from mice receiving a lethal dose of TAA (1000 mg/kg) had severe damage to liver tissue. Nobular disorganization and central vein (CV) hemorrhaging in the liver was detected 24 hours after administration of a lethal dose of TAA (FIG. 11B). Liver parenchyma from mice receiving a dose of TAA (500 mg/kg) also suffered from liver damage. Lymphocytic infiltration surrounding the CV was visible 3 days after ALF induction, indicative of parenchymal injury in this area (FIGS. 11C and 11D) Celastrol treatment following ALF resulted in the regression of liver injury. Liver parenchyma observed 3 days after ALF induction by TAA (500 mg/kg) and subsequent celastrol administration had primarily the appearance of normal liver tissue and negligible TAA induced damage. Celastrol treatment prior to or concurrent with induction of liver failure by TAA gave similar results as celastrol treatment post induction of liver failure. This analysis shows that celastrol rescued liver tissue in mice with ALF induced by TAA.

### Example 11

### Celastrol Increased Survival in a Mouse Model of ALF

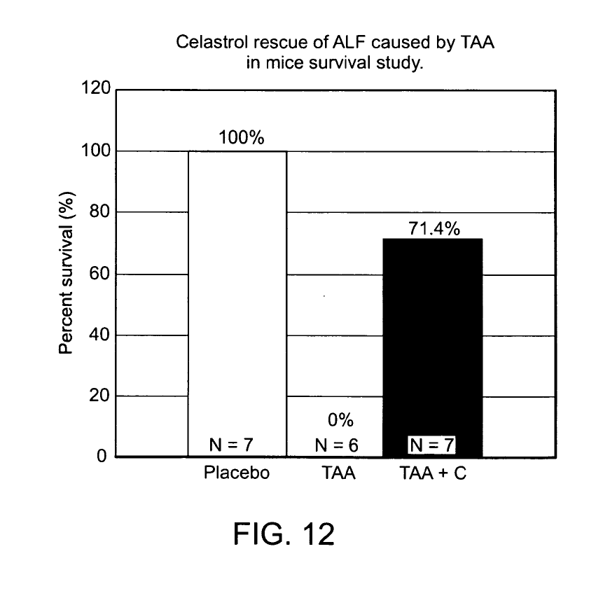

Thiomacetamide (TAA) was administered to C57BL6/J mice to induce liver damage. Groups of at least 6 C57BL6/J mice received either TAA (1000 mg/kg) and a placebo, TAA (1000 mg/kg) and celastrol (3 mg/kg), or placebo only by intraperitoneal injection. Mice were examined for 3 days or longer to determine survival (FIG. 12).

The administration of placebo had no effect on the mortality of mice. TAA caused 100% mortality in mice due to acute liver failure. Administration of celastrol to TAA treated mice resulted in 71.4% survival. Celastrol treatment prior to or concurrent with induction of liver failure by TAA gave similar results as celastrol treatment post induction of liver failure. This result indicated that celastrol is useful for the treatment of ALF.

### Example 12

### Celastrol Increased Survival in a Mouse Model of Heart Disease

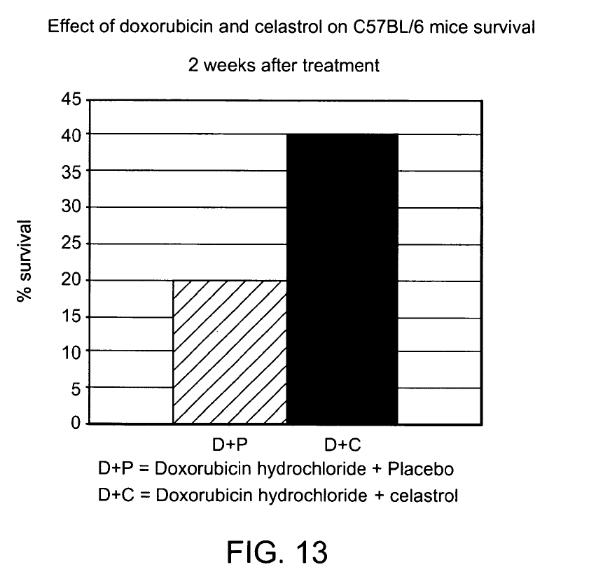

The administration of doxorubicin (DOX; adriamycin) to induce cardiac damage is a well-known model of heart disease see for example, Rosenhoff et al., “Adriamycin-induced cardiac damage in the mouse: a small-animal model of cardiotoxicity.” J Natl Cancer Inst. July 1975; 55(1):191-4; and van der Vijgh et al., “Morphometric study of myocardial changes during doxorubicin-induced cardiomyopathy in mice.” Eur J Cancer Clin Oncol. October 1988; 24(10):1603-8. Doxorubicin was administered to C57BL6/J mice to induce heart damage. Groups of 13-14 C57BL6/J mice received either DOX (20 mg/kg) and a placebo, or DOX (20 mg/kg) and celastrol (3 mg/kg). Mice were examined after 2 weeks to determine survival (FIG. 13).

The administration of doxorubicin with placebo caused 80% mortality in mice due to cardiac failure. Administration of celastrol to DOX treated mice resulted in 40% survival. This result indicated that celastrol is useful for the treatment of heart disease.

### Example 13

### Oridonin Mobilizes Bone Marrow Derived Stem Cells

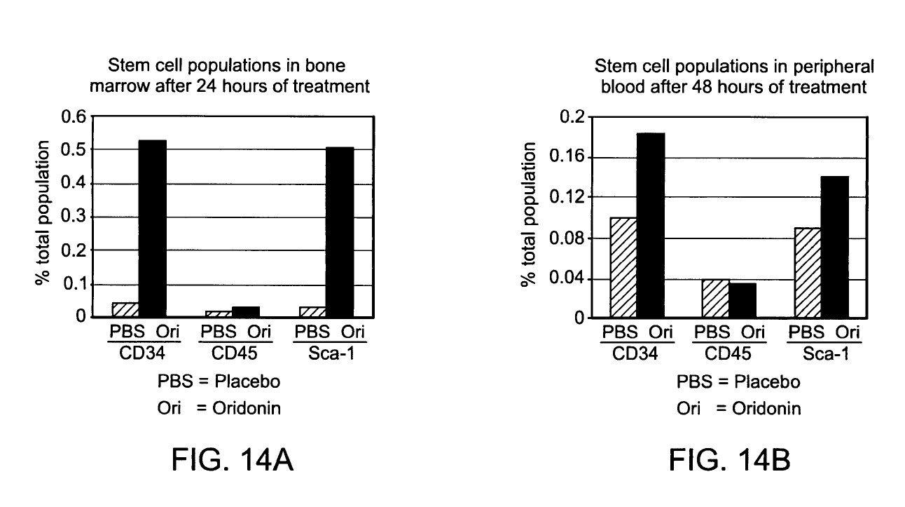

Oridonin activates stem cell populations in bone marrow and mobilizes them into peripheral blood in C57BL6/J mice as shown in FIGS. 14A and 14B. Oridonin was administered to C57BL6/J mice to determine the effect of oridonin on stem cell populations in bone marrow and in blood. Mice received either a placebo (PBS) or oridonin (3.0 mg/kg) by intraperitoneal injection. Bone marrow samples were taken from both groups of mice at 24 hours after administration of the placebo or oridonin. Blood samples were taken from both groups of mice at 48 hours after administration of the placebo or oridonin. The bone marrow samples and blood samples were analyzed by FACS for cells expressing stem cell markers. To observe the effect of treatment with oridonin, cells expressing the stem cell markers CD34, CD45, and Sca-1 in the bone marrow and blood samples, were quantitated as a percentage of the total populations in their respective samples (i.e., in the bone marrow sample and in the blood sample, respectively).

For the stem cell markers evaluated, bone marrow samples showed an increase in the population of cd34⁺ or Sca-1⁺ stem cells in mice treated with oridonin compared to samples from untreated mice (FIG. 14A). At 24 hours post treatment with oridonin, it was observed that CD34 cells represented greater than 0.5% of the population of bone marrow cells. This effect was at least a 10-fold increase over the percentage of cd34⁺ cells in bone marrow found in the control. Bone marrow from mice treated with oridonin also exhibited increases in the proportion of Sca-1⁺ stem cells in bone marrow compared to untreated mice. At 24 hours post treatment with oridonin, it was observed that Sca-1⁺ cells represented about 0.5% of the population of bone marrow cells. This effect was at least a 10-fold increase over the percentage of Sca-1⁺ cells in bone marrow found in the control. At 24 hours post treatment with oridonin, the cd45⁺ cell population did not change relative to that observed in the untreated control mice. This analysis shows that oridonin stimulate the increase of stem cell populations in bone marrow.

Blood samples also showed an increase in the population of cd34⁺ or Sca-1⁺ stem cells in mice treated with oridonin compared to samples from untreated mice (FIG. 14B). At 48 hours post treatment with oridonin, it was observed that cd34⁺ represented approximately 0.18% of the population of blood cells. This effect was about a 1.8-fold increase over the percentage of cd34⁺ cells in blood found in the control. Blood from mice treated with oridonin also exhibited increases in the proportion of Sca-1⁺ stem cells in blood compared to untreated mice. At 48 hours post treatment with oridonin, it was observed that Sca-1⁺ cells represented 0.14% of the population of blood cells. This effect was about a 1.4-fold increase over the percentage of Sca-1⁺ cells in blood found in the control. At 48 hours post treatment with oridonin, the Sca-1⁺ cell population did not change relative to that observed in the untreated control mice. This analysis shows that oridonin stimulates the increase of stem cell populations in blood.

### Example 14

### Derivatives of Tripterygium wilfordii

Tripterygium is a woody vine native to Eastern and Southern China, Korea, Japan, and Taiwan. In China this plant, known as lei kung teng or lei gong teng (“Thunder God Vine”), To date, over 380 secondary metabolites have been reported from Tripterygium species. Of these, 95% are terpenoids, including triptolide. As described herein, triptolide is surprisingly effective at mobilizing stem cells. Other derivatives of Tripterygium, alone or in various combinations, with or without triptolide, are expected to be equally effective. Terpenoids dominate the medicinal chemistry of Tripterygium, whose chemistry has been reviewed by Hegnauer (Hegnauer, R., 1964. In: Chemotaxonomie der Pflanzen, vol. 3. Birkha\ddot{}user, Basel, pp. 395-407; Hegnauer, R., 1989. In: Chemotaxonomie der Pflanzen, vol. 8. Birkha\ddot{}user, Basel, pp. 222-232, 704-705) and by Lu et al. Chemical constituents of Tripterygium wilfordii. Jiangsu Yiyao 13, 640-643 (Chem. Abstr. 108:183584).

The terpenoids are derived from C₅ isoprene units joined in a head-to-tail fashion. They are represented by (C₅)ₙ and are classified as hemiterpenes (C₅), monoterpenes (C₁₀), sesquiterpenes (C₁₅), diterpenes (C₂₀ such as triptolide and tripdiolide), sesterterpenes (C₂₅), triterpenes (C₃₀) and tetraterpenes (C₄₀). The active isoprene units that are synthesized into terpenoids are the diphosphate esters dimethylallyl diphosphate (DMAPP) and isopentenyl diphosphate (IPP). In higher plants, the biosynthesis of terpenoids proceeds via two independent pathways localized in different cellular compartments. The mevalonate (MVA) pathway in the cytoplasm is responsible for the biosynthesis of sesquiterpenes and triterpenes. Plastids contain the 1-deoxy-D-xylulose-5-phosphate (DOXP) pathway for the biosynthesis of monoterpenes, diterpenes, and tetraterpenes.

In the cytoplasm-localized MVA pathway, three molecules of acetyl-coenzyme A are used to produce MVA. Two ATP react with MVA to produce mevalonate diphosphate, followed by decarboxylation and dehydration with the involvement of a third molecule of ATP to give IPP. IPP is isomerized to the other isoprene unit, DMAPP, by isopentenyl-diphosphate-Disomerase (EC 5.3.3.2). IPP and DMAPP are active hemiterpene intermediates (C₅) in the pathways leading to more complicated terpenoids. DMAPP can produce the fundamental sesquiterpene precursor farnesyl diphosphate (FPP), with the successive addition of two furtherfurther IPPs. FPP can then give rise to a range of linear and cyclic sesquiterpenes. Two molecules of FPP are joined tail-to-tail to yield the precursor of triterpenes, squalene (C₃₀), from which other triterpenes arise.

In the plastid-localized DOXP pathway, pyruvate reacts with glyceraldehyde-3-phosphate (GA-3P) to yield DOXP. Then DOXP can form IPP through a series of reactions. IPP is isomerized to the other isoprene unit, DMAPP, by isopentenyl-diphosphate-D-isomerase (EC 5.3.3.2). Combination of DMAPP and IPP via the enzyme dimethylallytranstransferase (EC 2.5.1.1) produces a monoterpene diphosphate (C10), geranyl diphosphate (GPP). GPP can be isomerized to linalyl PP and neryl PP. These three compounds can produce a range of linear monoterpenes. The linear monoterpenes can create monocyclic and bicyclic systems via cyclization reactions. GPP can produce the fundamental diterpene precursor (C20), geranylgeranyl diphosphate (GGPP), with the successive additions of a further two IPPs (. Two molecules of GGPP are joined tail-to-tail to form a tetraterpene compound phytoene (C₄₀), a precursor for other tetraterpenes.

The two biosynthetic pathways of terpenoids are summarized below. The two terpenoid biosynthetic pathways are not totally independent. In cultured cells of the liverwort (Heteroscyphus planus), the cytoplasmic FPP was found to transfer into the plastid where FPP was condensed with a DOXPderived IPP. In snapdragon (Antirrhinum majus) flowers, the plastidal IPP transferred into the cytoplasm.

The structure of triptolide is shown in Formula 1, below.

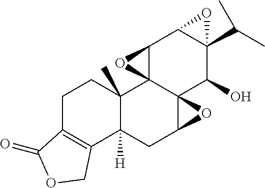

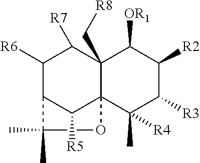

| | Compound name | R1 | R2 | R3 | R4 | R5 | R6 | R7 | R8 |
|---|---|---|---|---|---|---|---|---|---|
| 2 | triptofordin D-2 | Cin | H | H | OH | OAc | β-OAc | β-OBz | OAc |
| 3 | triptofordin E | Bz | OAc | H | OH | OAc | O (keto) | β-OBz | OAc |
| 4 | compound 8 | Ac | OAc | H | OH | OAc | O (keto) | β-OBz | OAc |
| 5 | triptofordin F-2 | Ac | OAc | H | OH | OH | α-OBz | β-OBz | OAc |
| 6 | triptogelin A-1 | Bz | OBz | H | H | OAc | β-OBz | β-OBz | H |
| 7 | triptogelin A-3 | H | OH | H | H | OAc | β-OBz | β-OBz | H |
| 8 | triptogelin C-1 | Ac | OAc | H | H | OAc | H | α-OBz | H |
| 9 | triptogelin G-1 | Ac | H | H | H | H | H | α-OCin | H |
| 10 | 1β-furanoyl-2β, 3α, 7α, 8β, 11- | Fur | OAc | OAc | OH | OH | α-OAc | β-OAc | OAc |
| | pentaacetoxy-4α,5α-dihydroxy- | | | | | | | | |
| | dihydroagarofuran | | | | | | | | |
| 11 | 1β, 2β, 3α, 5α, 7β, 8β, 11- | Ac | OAc | OAc | H | OAc | β-OAc | β-OAc | OAc |
| | heptaacetoxy- | | | | | | | | |
| | dihydroagarofuran | | | | | | | | |
| 12 | 1β-furanoyl-2β, 3α, 7α, 8β, 11- | Fur | OAc | OAc | H | OH | α-OAc | β-OAc | OAc |
| | pentaacetoxy-5α-hydroxy- | | | | | | | | |
| | dihydroagarofuran | | | | | | | | |
| 13 | 1β, 7β, 8α-triacetoxy-2β- | Ac | OFur | H | OH | OAc | β-OAc | α-OAc | OCOCH(Me)2 |
| | furanoyl-4α-hydroxy-11- | | | | | | | | |
| | isobutyryloxy- | | | | | | | | |
| | dihydroagarofuran | | | | | | | | |
| 14 | 1β-nicotinoyl-2β, 5α, 7β- | Nic | OAc | H | OH | OAc | β-OAc | α-OFur | OCOCH(Me)2 |
| | triacetoxy-4α-hydroxy-11- | | | | | | | | |
| | isobutyryloxy-8α-furanoyl- | | | | | | | | |
| | dihydroagarofuran | | | | | | | | |

Formulas 2-14 are bioactive dihydroagarofurans in Tripterygium. The abbreviations used have the following meanings: Ac=acetate, Cin=cinnamoyl, Bz=benzoyl, Fur=furanoyl, Nic=nicotinoyl.

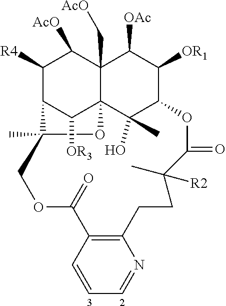

| | Compound name | R1 | R2 | R3 |
|---|---|---|---|---|
| 15 | wilfortrine | Fur | OH | Ac |
| 16 | wilforine | Bz | H | Ac |
| 17 | wilfordine | Bz | OH | Ac |
| 18 | wilforgine | Fur | H | Ac |
| 19 | wilforidine | H | OH | Ac |
| 20 | wilfornine (= 2-debenzoyl-2- | Nic | H | Ac |
| | nicotinoyl-wilforine) | | | |
| 21 | euonine (= wilformine) | Ac | H | Ac |
| 22 | alatusinine | Ac | OH | Ac |

Formulas 15-22 are Wilforine-type active sesquiterpene alkaloids in Tripterygium. The abbreviations used have the following meanings: Ac=acetate, Bz=benzoyl, Fur=furanoyl, Nic=nicotinoyl.

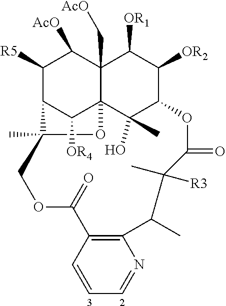

| | Compound name | R₁ | R₂ | R₃ | R₄ | R₅ |
|---|---|---|---|---|---|---|
| 23 | euonymine | Ac | Ac | H | Ac | OAc |
| 24 | wilfordsine (N at pos. 3) | Ac | Bz | OH | Ac | OAc |
| 25 | cangorinine E-1 | Ac | Ac | H | Bz | OAc |
| 26 | mayteine | Bz | Ac | H | Ac | OAc |
| 27 | ebenifoline E-II | Bz | Ac | H | Bz | OAc |
| 28 | wilfordconine (N at pos. 3) | Ac | H | OH | Ac | OFur |

Formulas 23-28 are Euonymine-type active sesquiterpene alkaloids in Tripterygium.

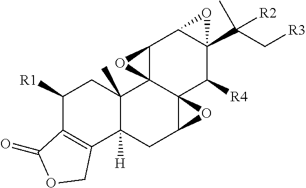

| | Compound name | R1 | R2 | R3 | R4 |
|---|---|---|---|---|---|
| 30 | triptonide | H | H | H | O (keto) |
| 31 | tripdiolide | OH | H | H | OH |
| 32 | triptolidenol | H | OH | H | OH |
| 33 | 16-hydroxytriptolide | H | H | OH | OH |

Formulas 30-33 are Bioactive triptolide derivatives in Tripterygium.

| 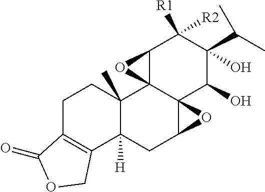 | | | |
|---|---|---|---|
| | Compound name | R₁ | R₂ |
| 34 | triptriolide | OH | H |
| 35 | 12-epitriptriolide | H | OH |
| 36 | tripchlorolide | Cl | H |

Formulas 34-36 are Diterpene diepoxides in Tripterygium.

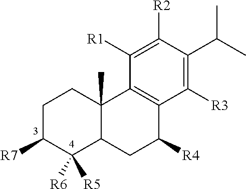

| | Compound name | R₁ | R₂ | R₃ | R₄ | R₅ | R₆ | R₇ |
|---|---|---|---|---|---|---|---|---|
| 38 | dehydroabietic | H | H | H | H | Me | COOH | H |
| | acid | | | | | | | |
| 39 | triptobenzene H | OH | H | OMe | H | Me | none | COOH |
| | (dbl bond C3-4) | | | | | | | |
| | (= hypoglic acid) | | | | | | | |
| 40 | triptoditerpenic | H | H | OMe | H | Me | none | COOH |
| | acid B | | | | | | | |
| | (dbl bond C3-4) | | | | | | | |
| | (= triptinin-A) | | | | | | | |
| 41 | (+)-dehydro- | H | H | H | H | Me | Me | H |
| | abietane | | | | | | | |
| | (= abietatriene) | | | | | | | |
| 42 | abieta-8,11,13- | H | H | H | O | Me | Me | H |
| | trien-7-one | | | | (keto) | | | |
| 43 | triptenin B | H | H | OH | H | Me | none | COOH |
| | (dbl bond C3-4) | | | | | | | |
| | (= triptinin-B) | | | | | | | |
| 44 | triptobenzene J | H | H | OH | H | CH₂OH | Me | OH |
| 45 | hinokiol | H | OH | H | H | Me | Me | OH |

Formulas 38-45 are Bioactive benzenoid abietanes from Tripterygium.

Formula 46, which is quinone, follows:

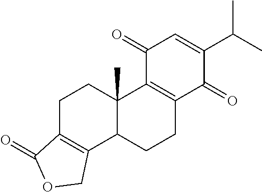

| 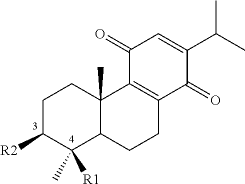 | | | |
|---|---|---|---|
| | Compound name | R₁ | R₂ |
| 47 | triptoquinone A (dbl bond C3-4) | none | COOH |
| | (= triptoquinonoic acid A) | | |
| 48 | triptoquinone B | CH₂OH | O (keto) |
| 49 | triptoquinone C | CH₂OH | OH |
| | (= triptoquinondiol) | | |
| 50 | triptoquinone D (= triptoquinonol) | CH₂OH | H |
| 51 | triptoquinone E (= triptoquinonal) | CHO | H |
| 52 | triptoquinone F | COOH | H |
| | (= triptoquinonoic acid B) | | |
| 53 | triptoquinone H | Me | O (keto) |

Formulas 47-53 are bioactive diterpene quinoids from Tripterygium.

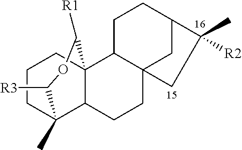

| | Compound name | R₁ | R₂ | R₃ |
|---|---|---|---|---|
| 54 | tripterifordin (= hypodiofide A, | HO | HO | (keto) |
| | antriptolactone | | | |
| 55 | 16α-hydroxy-19,20-epoxy-19R- | H | OH | OEt |
| | ethoxy-kaurane | | | |
| 56 | 16α-hydroxy-19,20-epoxy-20R- | OEt | OH | H |
| | ethoxy-kaurane | | | |
| 57 | doianoterpene A (dbl bond C15-16) | O (keto) | none | H |

Formulas 54-57 are bioactive five-ring kauranes from Tripterygium.

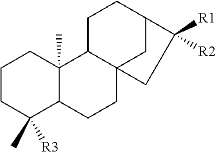

| | Compound name | R₁ | R₂ | R₃ |
|---|---|---|---|---|
| 58 | (−)-16α-hydroxy-kauran-19- | Me | OH | COOH |
| | oic acid | | | |
| 59 | (−)-17-hydroxy-16α-kauran- | CH₂OH | H | COOH |
| | 19-oic acid | | | |
| 60 | 16α-(−)-kauran-17,19-dioic | H | COOH | COOH |
| | acid | | | |
| 61 | ent-19-hydroxy-kaur-16-en | vinyl | | CH₂OH |
| | (= ent-kaurenol) | | | |

Formulas 58-61 are bioactive four-ring kauranes from Tripterygium.

Formula 62 follows:

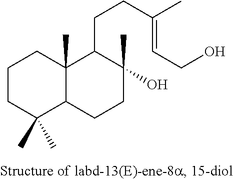

Formula 63 follows:

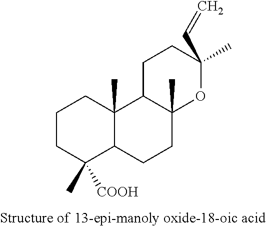

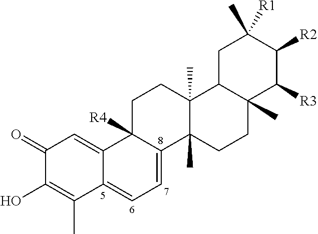

| | Compound name | R₁ | R₂ | R₃ | R₄ |
|---|---|---|---|---|---|
| 64 | pristimerin | COOCH₃ | H | H | Me |
| 65 | celastrol (= tripterin) | COOH | H | H | Me |
| 66 | tingenone (= tingenin A, | H | O (keto) | H | Me |
| | maitenin, maytenin) | | | | |
| 67 | 22β-hydroxy-tingenone | H | O (keto) | OH | Me |
| | (= tingenin B) | | | | |
| 68 | tripterygone (no dbl bonds | COOH | H | H | H |
| | C5-6 and 7-8; β-Me at C5) | | | | |

Formulas 64-68 are bioactive quinone methides from Tripterygium.

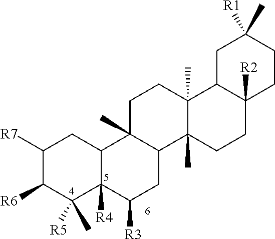

| | Compound name | R₁ | R₂ | R₃ | R₄ | R₅ | R₆ | R₇ |
|---|---|---|---|---|---|---|---|---|
| 69 | polpunonic acid | COOH | Me | H | Me | H | O | H |
| | (= maytenoic acid, | | | | | | (keto) | |
| | maytenonic acid) | | | | | | | |
| 70 | 3-oxo-friedelan- | Me | COOH | H | Me | H | O | H |
| | 28-oic acid | | | | | | (keto) | |
| 71 | 3β, 29- | CH₂OH | Me | H | none | Me | OH | H |
| | dihydroxy- | | | | | | | |
| | D:B-friedoolean- | | | | | | | |
| | 5-en | | | | | | | |
| | (dbl bond C5-6) | | | | | | | |
| 72 | wilforic acid B | COOH | Me | H | none | none | O | β-OH |
| | (dbl bond C4-5) | | | | | | (keto) | |
| 73 | regeol B | COOH | Me | OH | none | none | O | α-OH |
| | (dbl bond C4-5) | | | | | | (keto) | |
| 74 | 29-hydroxy- | CH₂OH | Me | H | Me | H | O | H |
| | friedelan-3-one | | | | | | (keto) | |
| | (= D:A- | | | | | | | |
| | friedooleanan- | | | | | | | |
| | 29-ol-3-one) | | | | | | | |

Formulas 69-74 are bioactive five-ring friedelanes/friedooleananes with saturated rings from Tripterygium.

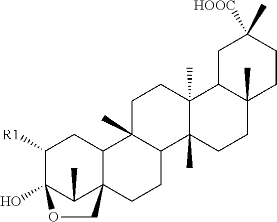

| | Compound name | R1 |
|---|---|---|
| 75 | orthosphenic acid | OH |
| 76 | salaspermic acid | H |

Formulas 75 and 76 are bioactive six-ring friedelanes/friedooleananes with saturated rings from Tripterygium.

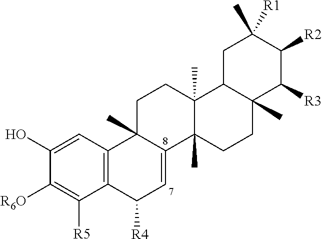

| | Compound | | | | | | |
|---|---|---|---|---|---|---|---|
| | name | R₁ | R₂ | R₃ | R₄ | R₅ | R₆ |
| 77 | demethyl- | COOH | H | H | O (keto) | CHO | H |
| | zeylasteral | | | | | | |
| 78 | demethyl- | COOH | H | H | O (keto) | COOH | H |
| | zeylasterone | | | | | | |
| 79 | wilforic acid A | COOH | H | H | H | Me | H |
| | (no dbl bond | | | | | | |
| | at C7-8) | | | | | | |
| 80 | triptohypol C | COOH | H | H | H | Me | H |
| 81 | 3-methyl-22β, | H | O | OH | O (keto) | CH₂OH | Me |
| | 23-diol-6- | | (keto) | | | | |
| | oxotingenol | | | | | | |
| 82 | 2,3-dihydroxy- | COOH | H | H | CH(OH)—Me | Me | H |
| | 1,3,5(10),7- | | | | | | |
| | tetraene-6α(1′- | | | | | | |
| | hydroxyethyl)- | | | | | | |
| | 24-nor-D:A- | | | | | | |
| | friedooleane- | | | | | | |
| | 29-oic acid | | | | | | |

Formulas 77 and 82 are bioactive friedooleananes with a benzenoid ring from Tripterygium.

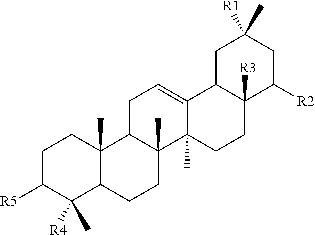

| | Compound name | R₁ | R₂ | R₃ | R₄ | R₅ |
|---|---|---|---|---|---|---|
| 83 | oleanolic acid | Me | H | COOH | Me | β-OH |
| 84 | 3-acetoxy-oleanolic acid | Me | H | COOH | Me | β-OAc |
| 85 | triptotriterpenic acid A | COOH | α-OH | Me | Me | β-OH |
| | (= abrusgenic acid, | | | | | |
| | maytenfolic acid) | | | | | |
| 86 | 3-epikatonic acid | COOH | H | Me | Me | β-OH |
| 87 | triptotriterpenic acid B | COOH | β-OH | Me | Me | β-OH |
| 88 | β-amyrin | Me | H | Me | Me | β-OH |
| 89 | triptotriterpenonic | COOH | α-OH | Me | Me | O |
| | acid A (= 22α- | | | | | (keto) |
| | hydroxy-3-oxo-olean- | | | | | |
| | 12-en-29-oic acid) | | | | | |
| 90 | katononic acid | COOH | H | Me | Me | O |
| | | | | | | (keto) |
| 91 | wilforol C | Me | H | COOH | CH₂OH | α-OH |
| 92 | triptocallic acid D | COOH | α-OH | Me | Me | α-OH |

Formulas 83-92 are bioactive five-ring oleananes from Tripterygium.

Formulas 93-95 are Bioactive six-ring oleananes from Tripterygium.

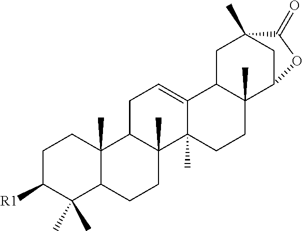

| | Compound name | R₁ |
|---|---|---|
| 93 | regelide (= wilforlide A, abruslactone A) | OH |
| 94 | wilforlide B | O (keto) |
| 95 | 2α, 3β-dihydroxy-olean-12-ene-22,29-lactone | OH |

Formulas 96-104 are bioactive ursanes from Tripterygium.

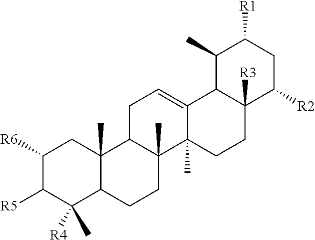

| | Compound name | R₁ | R₂ | R₃ | R₄ | R₅ | R₆ |
|---|---|---|---|---|---|---|---|
| 96 | regelin | COOMe | OH | Me | Me | O | H |
| | | | | | | (keto) | |
| 97 | regelinol | COOMe | OH | Me | CH₂OH | O | H |
| | | | | | | (keto) | |
| 98 | triptotriterpenic acid C | COOH | OH | Me | Me | β-OH | H |
| | (= tripterygic acid A) | | | | | | |
| 99 | α-amyrin | Me | H | Me | Me | β-OH | H |
| 100 | triptocallic acid A | COOH | OH | Me | Me | α-OH | H |
| 101 | dulcioic acid | COOH | H | Me | Me | β-OH | H |
| 102 | demethylregelin | COOH | OH | Me | Me | O | H |
| | | | | | | (keto) | |
| 103 | 3β-acetoxy-ursolic acid | Me | H | COOH | Me | β-OAc | H |
| | (= acetyl ursolic acid) | | | | | | |
| 104 | 2α-hydroxy-ursolic acid | Me | H | COOH | Me | β-OH | OH |
| | (= corosolic acid, | | | | | | |
| | colosolic acid) | | | | | | |

Formulas 105-106 are bioactive steroids from Tripterygium.

| 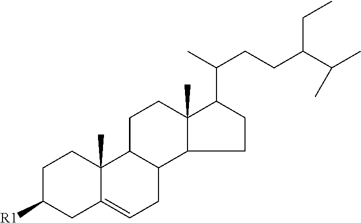 | | |
|---|---|---|
| | Compound name | R₁ |
| 105 | β-sitosterol | OH |
| 106 | daucosterol | O-β-D-glucopyranose |

If desired, such agents are administere to a subject at 50, 100, 200, 250, 300, 350, or 500 μg/kg.

### Example 15

### Valproic Acid Mobilizes Bone Marrow Derived Stem Cells

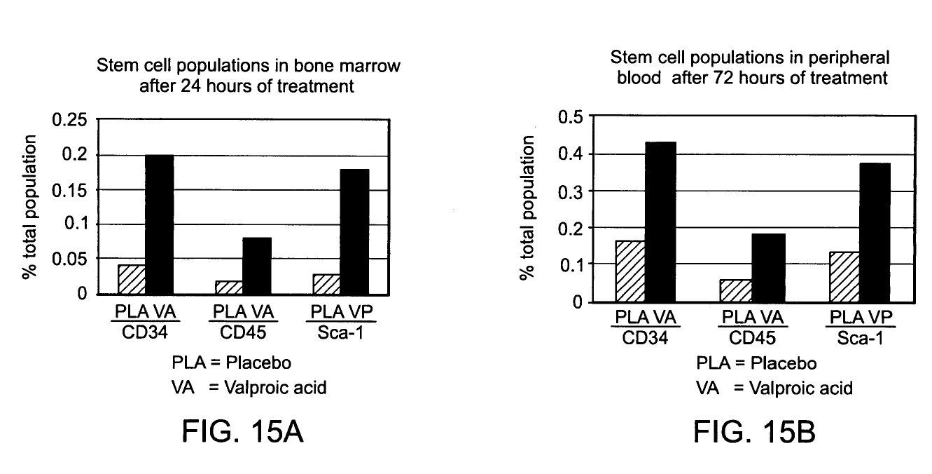

Valproic acid activates stem cell populations in bone marrow and mobilizes them into peripheral blood in C57BL6/J mice as shown in FIGS. 15A and 15B. Valproic acid was administered to C57BL6/J mice to determine the effect of valproic acid on stem cell populations in bone marrow and in blood. Mice received either a placebo or valproic acid (200 mg/kg) by intraperitoneal injection. Even at this dose, valproic acid was not toxic in mice. Bone marrow samples were taken from both groups of mice at 24 hours after administration of the placebo or valproic acid. Blood samples were taken from both groups of mice at 72 hours after administration of the placebo or valproic acid. The bone marrow samples and blood samples were analyzed by FACS for cells expressing stem cell markers. To observe the effect of treatment with valproic acid, cells expressing the stem cell markers CD34, CD45, and Sca-1 in the bone marrow and blood samples, were quantitated as a percentage of the total populations in their respective samples (i.e., in the bone marrow sample and in the blood sample, respectively).

For the stem cell markers evaluated, bone marrow samples showed an increase in the population of cd34⁺, cd45⁺, and Sca-1⁺ stem cells in mice treated with valproic acid compared to samples from untreated mice (FIG. 15A). At 24 hours post treatment with valproic acid, it was observed that CD34 cells represented about 0.2% of the population of bone marrow cells. This effect was at least a 4-fold increase over the percentage of cd34⁺ cells in bone marrow found in the control. Bone marrow from mice treated with valproic acid also exhibited increases in the proportion of cd45⁺ and Sca-1⁺ stem cells in bone marrow compared to untreated mice. At 24 hours post treatment with valproic acid, it was observed that cd45⁺ cells represented about 0.08% of the population of bone marrow cells. This effect was at least about a 3-fold increase over the percentage of cd45⁺ cells in bone marrow found in the control. At 24 hours post treatment with valproic acid, it was observed that Sca-1⁺ cells represented about 0.18% of the population of bone marrow cells. This effect was about a 6-fold increase over the percentage of Sca-1⁺ cells in bone marrow found in the control. This analysis shows that valproic acid stimulate the increase of stem cell populations in bone marrow.

Blood samples also showed an increase in the population of cd34⁺ or Sca-1⁺ stem cells in mice treated with valproic acid compared to samples from untreated mice (FIG. 15B). At 72 hours post treatment with valproic acid, it was observed that cd34⁺ represented greater than 0.4% of the population of blood cells. This effect was about a 2.6-fold increase over the percentage of cd34⁺ cells in blood found in the control. Blood from mice treated with valproic acid also exhibited increases in the proportion of cd45⁺ and Sca-1⁺ stem cells in blood compared to untreated mice. At 72 hours post treatment with valproic acid, it was observed that cd45⁺ cells represented a little less than 0.2% of the population of blood cells. This effect was almost a 4-fold increase over the percentage of cd45⁺ cells in blood found in the control. At 72 hours post treatment with valproic acid, it was observed that Sca-1⁺ cells represented a little less than 0.4% of the population of blood cells. This effect was about a 3-fold increase over the percentage of Sca-1⁺ cells in blood found in the control. This analysis shows that valproic acid stimulates the increase of stem cell populations in blood.

### Example 16

### Celastrol Ameliorates Diabetes in NOD Mouse Model

The NOD mouse is recognized as a model for human Type1 diabetes (Anderson and Bluestone, “THE NOD MOUSE: A Model of Immune Dysregulation” Annual Review of Immunology 23: 447-485, 2005, which is incorporated by reference in its entirety). Celastrol was administered to the mice at the rate of 3.0 mg/kg body weight via intra-peritoneal injection once a week in NOD mice starting from 4 weeks of age. When mice attained 11 weeks of age, the compound was administered at the rate of 3.3 mg/kg body weight via oral gavage three times a week up to 30 weeks of age. Blood glucose levels were measured at the time of celastrol administration once a week from 4-10 weeks and three times a week from 11-30 weeks. Mice in control group were treated with placebo.

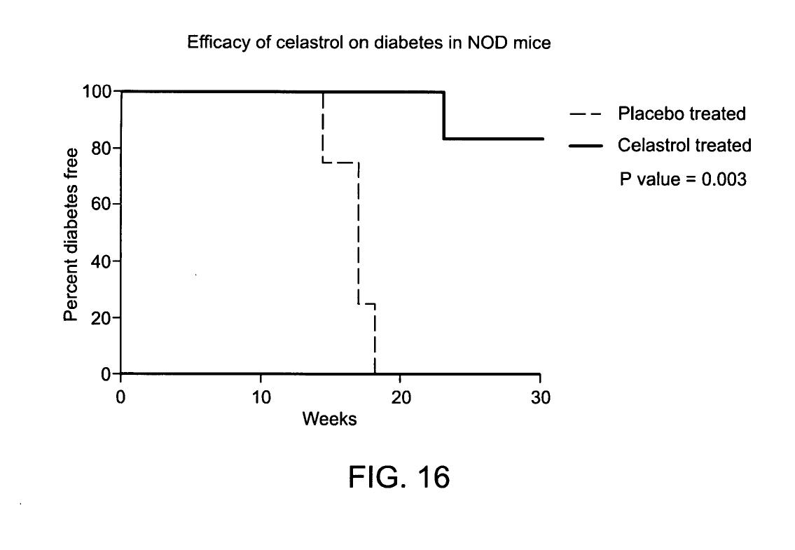

All mice in the control group (n=4) became hyperglycemic (more than 250 mg/dL) between 14.4 and 18.1 weeks of age. Surprisingly, only twenty percent (⅕) of celastrol treated mice became hyperglycemic at 23 weeks of age. Eighty percent of mice were euglycemic or normoglycemic (less than 250 mg/dL) up to 30 weeks of age (FIG. 16, Kaplan-Meier graph).

### Example 17

### Adaptive Transfer Experiment

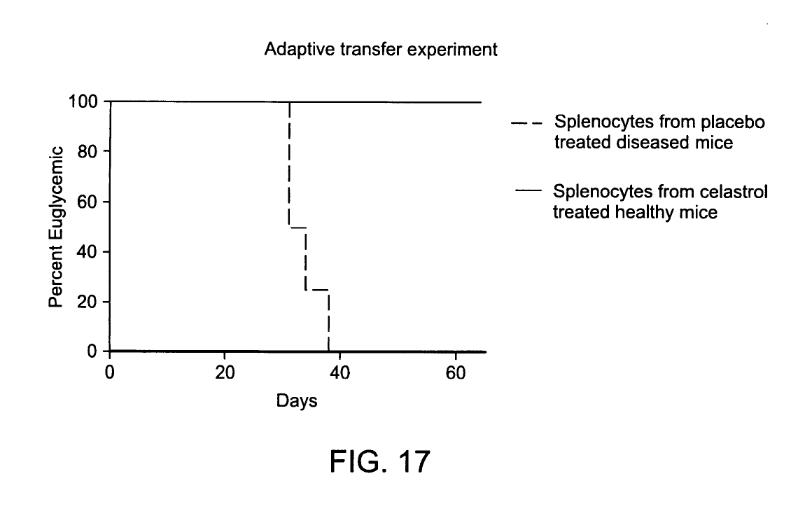

NOD mice were sacrificed and splenocytes collected from celastrol-treated healthy mice and mice that became diabetic recently were adaptively transferred at 5.3×10⁶ cells per mouse into NOD-scid mice (mice free from immune system) via tail vein injection. Blood glucose levels of the mice were monitored starting from 4 weeks after the adaptive transfer was made. All mice (n=4) that received splenocytes from diabetic mice became diabetic between 31-38 days (FIG. 17, Kaplan-Meier graph). In contrast, mice (12/12) that received splenocytes from celastrol treated mice were free of diabetes (hyperglycemic condition) at 68 day (FIG. 17). Without wishing to be bound by theory, these results indicate that celastrol may be acting, at least in part, to modulate the disregulated immune response causing diabetes in NOD mice.

### Example 18

### Celastrol Activates Bone Marrow Stem Cells and Cell Mobilization into Peripheral Blood Stream

As reported herein, celastrol activated and mobilized stem cells into peripheral blood. These observations were extended using GFP mice to analyse stem cell mobilization. Mice expressing GFP in their bone marrow were treated with celastrol and placebo. Blood samples were collected on the third day and red blood cells were lysed. White blood cells containing mobilized stem cells, if any, were injected at 0.5×10⁶ cells per mouse through retro-orbital sinus into wild type mice, C57BL/6 that were irradiated to destroy the hematopoietic systems. Cells obtained from placebo-treated GFP mice were injected into a group of wild type mice which served as control. Blood samples from wild type mice that received stem cells from celastrol-treated GFP mice were analyzed after 3, 4 and 5 months of transfer.

Mice that received cells from placebo treated GFP mice died between 12 and 16 days of transfer (n=7). Mice that received cells from celastrol treated GFP mice survived longer. Fifty percent of the mice survived for at least 5 months (n=4), and the other two mice survived for 17 and 25 days. Blood sample analysis of these mice showed GFP positive cells indicating that these cells came from celastrol-treated GFP mice and the mobilized stem cells were responsible for the survival of 50% mice.

### Example 19

### Secondary Adaptive Transfer

Wild type mice that survived the primary transfer were used for secondary transfer. Bone marrow cells obtained from these mice were collected and transferred into irradiated wild type mice, C57BL/6 at 1.5×10⁶ cells per mouse.

All the mice in control group died within 10 days of transfer after secondary transfer, whereas 85.7% mice, that received bone marrow cells from celastrol treated primary adaptive transfer group, were still surviving secondary transfer at 7 weeks. The observation is being continued. This indicates that the transferred bone marrow cells were able to restore the hematopoietic system of the irradiated mice.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. All references are incorporated by reference in their entirety. All patents and publications mentioned in this specification are herein incorporated by reference to the same extent as if each independent patent and publication was specifically and individually indicated to be incorporated by reference.

1. A method for tissue repair or regeneration, the method comprising contacting a cell with an effective amount of an agent having at least two activities selected from the group consisting of i) inhibition of hsp-90 biological activity; ii) mobilization of a bone marrow derived stem cell; iii) inhibition of apoptosis; and iv) modulation of an immune response.

2. The method of claim 1 for tissue repair or regeneration, the method comprising contacting a cell with an effective amount of an agent selected from the group consisting of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or structural or functional analogs or derivatives of said agents.

3.-12. (canceled)

13. The method of any of claims 1-2, wherein the method involves administering celastrol and triptolide, celastrol and TE-140, or celastrol and geldanamycin.

14. (canceled)

15. The method of any of claims 1-2, wherein the method further comprises identifying a subject as having a disease or disorder characterized by an undesirable increase in cell death or a deficiency in cell number.

16. A pharmaceutical composition for tissue repair or regeneration comprising an effective amount of an agent having at least two activities selected from the group consisting of i) inhibition of hsp-90 biological activity; ii) mobilization of a bone marrow derived stem cell; iii) inhibition of apoptosis; and iv) modulation of an immune response in a pharmaceutically acceptable excipient.

17. The pharmaceutical composition of claim 16 for tissue repair or regeneration comprising an effective amount of an agent selected from the group consisting of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or functional or structural analogs thereof, in a pharmaceutically acceptable excipient.

18. The composition of claim 16 or 17, wherein the composition is labeled for use in tissue repair or regeneration.

19.-27. (canceled)

28. A method of activating or mobilizing a bone marrow derived cell in a subject in need thereof, the method comprising
(a) administering to the subject an effective amount of an agent selected from the group consisting of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and structural or functional analogs thereof; and
(b) administering an effective amount of TE-140 peptide, GM-CSF and/or Stem Cell Factor, wherein the amount of said agent and GM-CSF and/or Stem Cell Factor is sufficient to activate or mobilize a bone marrow derived cell in the subject.

29. The method of claim 28, wherein the method is useful for the treatment or prevention of a disease or disorder characterized by increased cell death or a deficiency in cell number.

30. The method of claim 28, wherein the subject is in need of tissue repair or regeneration.

31. The method of claim 28, wherein the tissue in need of repair is selected from the group consisting of bladder, blood system, bone, breast, cartilage, esophagus, fallopian tube, gall bladder, glial cell, heart, intestines, kidney, lung, lymphatic system, muscle, ovaries, pancreas, prostate, spleen, stomach, testes, thymus, thyroid, trachea, urogenital tract, ureter, urethra, uterus, skeletal muscle, and skin.

32. The method of claim 28, wherein the method is useful for the treatment or prevention of diabetes, acute liver failure, myocardial infarction, heart failure, cardiomyopathy, lung disease, wounding, hematopoietic cell loss related to radiation or chemotherapeutic ablation, or trauma-induced injury.

33. The method of claim 28, wherein the bone marrow derived cell is a hematopoietic stem cell.

34. The method of claim 28, wherein the agent is administered locally or systemically.

35.-43. (canceled)

44. A method of inhibiting pancreatic cell death in a subject, the method comprising contacting a pancreatic cell at risk of cell death with an agent having at least two activities selected from the group consisting of i) inhibition of hsp-90 biological activity; ii) mobilization of a bone marrow derived stem cell; iii) inhibition of apoptosis; and iv) modulation of an immune response.

45. The method of claim 44, wherein the agent is triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or structural or functional analogs thereof.

46. (canceled)

47. A method of treating or preventing diabetes in a subject, the method comprising administering an effective amount of a compound selected from the group consisting of triptolide, a Tryptigerium derivative of Formula 1-106, oridonin, geldanamycin, celastrol, dihydrocelastrol, dihydrocelastrol diacetate, pristimerol, 17-AAG, oridonin, valproic acid, a combination of celastrol and geldanamycin, a combination of celastrol and 17-AAG, a combination of celastrol and triptolide, and a combination of celastrol and a TE-140 peptide, or structural or functional analogs, thereby treating or preventing diabetes.

48. The method of claim 44, wherein the method reduces pancreatic cell death by at least 10% relative to the level in an untreated reference.

49. The method of claim 47, wherein the subject has type I or type II diabetes.

50.-52. (canceled)

53. The method of claim 49, wherein the administration increases insulin production.

54.-92. (canceled)
